(19) United States
(12) Patent Application Publication
(10) Pub. No.: US 2026/0158172 A1
(43) Pub. Date: Jun. 11, 2026

(54) CELL DEATH METHOD USING CRISPR/CAS SYSTEM HAVING SINGLE-STRAND BREAK ACTIVITY AND PARP INHIBITOR

(71) Applicants: INSTITUTE FOR BASIC SCIENCE, Daejeon (KR); UNIST(ULSAN NATIONAL INSTITUTE OF SCIENCE AND TECHNOLOGY), Ulsan (KR)

(72) Inventors: Seung Woo CHO, Ulsan (KR); So Young LEE, Ulsan (KR); Taejoon KWON, Ulsan (KR); Kyungjae MYUNG, Ulsan (KR)

(21) Appl. No.: 19/535,401

(22) Filed: Feb. 10, 2026

Related U.S. Application Data
(63) Continuation of application No. PCT/KR2024/012084, filed on Aug. 13, 2024.

(30) Foreign Application Priority Data
Aug. 16, 2023 (KR) 10-2023-0107089

(51) Int. Cl. A61K 48/00; A61K 31/166; A61K 31/4184; A61K 31/454; A61K 31/502; A61K 31/55; A61K 38/46; A61P 35/00

(52) U.S. Cl. CPC A61K 48/0058; A61K 31/166; A61K 31/4184; A61K 31/454; A61K 31/502; A61K 31/55; A61K 38/465; A61P 35/00

(57) ABSTRACT

Disclosed herein is a cell death method using a CRISPR/Cas system having single-strand break activity together with a PARP inhibitor, as one type of cell death method using a CRISPR/Cas system. This method is a safer cell death technology with few unwanted side effects, compared to cell death methods using CRISPR/Cas systems having double-strand break activity. Disclosed herein are a method for treating cancer by using the cell death method, and specific configurations required for implementing the methods.

### CROSS-REFERENCE TO RELATED APPLICATIONS

This application is a continuation application of PCT Application No. PCT/KR2024/012084, filed on 13 Aug. 2024, which claims priority to Korean Patent Application No. 10-2023-0107089, filed on 16 Aug. 2023. The entire disclosure of the applications identified in this paragraph is incorporated herein by reference.

### SEQUENCE LISTING

This application contains references to amino acid sequences and/or nucleic acid sequences which have been submitted concurrently herewith as the sequence listing XML file entitled “000111uscoa_SequenceListing. XML”, file size 73,728 bytes, created on 10 Feb. 2026. The aforementioned sequence listing is hereby incorporated by reference in its entirety pursuant to 37 C.F.R. § 1.52(e)(5).

### FIELD

This specification relates to a technique for inducing cell death by cleaving multiple sites in the genome of a cell using a CRISPR/Cas system. Specifically, this specification relates to a target-specific cleavage technique using a CRISPR/Cas system and to a technique for inhibiting single-strand break repair in the genome of a cell using a PARP inhibitor.

### BACKGROUND

International Patent Publication No. WO 2020/055187 A1 discloses a cell death method using a CRISPR/Cas system having double-strand break activity. Specifically, the above document in the related art discloses a method for killing cancer cells by delivering a plurality of CRISPR/Cas systems targeting cancer cell-specific nucleic acid sequences to cells. However, CRISPR/Cas systems suffer from off-target issues in which sequences similar to a target nucleic acid sequence are erroneously recognized as targets and cleaved. For this reason, concerns are raised regarding undesired side effects in non-target cells. Accordingly, there is a need for a safer cell death technique with fewer undesired side effects for therapeutic development of CRISPR/Cas systems.

### Patent Document

International Patent Publication No. WO 2020/055187 A1

### SUMMARY

### Technical Problem

### Known Cell Death Methods Using CRISPR/Cas Systems Having Double-Strand Break Activity

International Patent Publication No. WO 2020/055187 A1 discloses a cell death method using a CRISPR/Cas system having double-strand break activity. Specifically, the above document in the related art discloses a method for killing cancer cells by delivering a plurality of CRISPR/Cas systems targeting cancer cell-specific nucleic acid sequences to cells. According to the above document in the related art, when a predetermined number or more of target nucleic acids within the genome of the cells are targeted using CRISPR/Cas systems and cleaved, this induces cell death.

### Problems Caused by Side Effects Due to Off-Target Activity of CRISPR/Cas System

CRISPR/Cas systems function in a target-specific manner according to a protospacer adjacent motif (PAM) sequence and guidance of guide RNA. However, it has been reported that CRISPR/Cas systems may erroneously recognize sequences similar to a target nucleic acid sequence as targets and cleave such sequences. This phenomenon is referred to as an off-target issue in CRISPR/Cas systems. When an off-target issue arises, unintended nucleic acids are cleaved. When such nucleic acids are present in the genome of cells, mutations such as indels are highly likely to be introduced. Such off-target issues are known as one of the significant side effects of CRISPR/Cas systems.

Need for Development of Safer Cell Death Technology with Fewer Undesired Side Effects

When the off-target issue in the CRISPR/Cas systems described above occurs, there are the following problems: 1) it is impossible to predict which nucleic acids will be adversely affected; and 2) it is entirely unknown what effects the resulting outcomes will have on a patient. Such problems constitute weaknesses when the CRISPR/Cas systems are used as therapeutic agents administered to patients, which must be overcome. Accordingly, there is a need for a safer cell death technique with fewer undesired side effects for therapeutic development.

### Technical Solution

The present specification discloses a method for killing a cell using a CRISPR/Cas system having single-strand break activity, together with a PARP inhibitor, as a solution to the aforementioned technical problems.

The present specification also discloses a method for treating cancer by applying the above cell death method.

The present specification also discloses specific configurations required for implementing the method for killing a cell and the method for treating cancer above.

### Advantageous Effects

By utilizing the method for killing a cell presented herein, cell death can be induced with an efficiency equivalent to or greater than that of a cell death method using a CRISPR/Cas system having double-strand break activity. Furthermore, using the method for killing a cell causes fewer side effects compared to when utilizing the CRISPR/Cas system having double-strand break activity. Specifically, the above side effects include the following problems: 1) when the CRISPR/Cas system is introduced into non-target cells, the system functions on non-target sequences, thereby introducing undesired mutations; and 2) cytotoxicity is observed toward non-target cells.

### BRIEF DESCRIPTION OF DRAWINGS

FIG. 1 shows experimental results according to Experimental Example 2. Specifically, the results were obtained by treating Hela cells with each of the substances listed in Table 1. Here, Olaparib (−) indicates a group not administered with a PARP inhibitor, and Olaparib (+) indicates a group administered with a PARP inhibitor. Additionally, Mock indicates a group administered with phosphate-buffered saline (PBS) only; NT indicates a group comprising a random sgRNA that does not target any sequence; Umix-4 indicates a group comprising sgRNAs targeting four target nucleic acids commonly comprised in the genomes of both Hela cells and HCT-116 cells; Hmix-4 indicates a group comprising sgRNAs targeting four target nucleic acids comprised only in the genome of HCT-116 cells; and MT-50 indicates a group comprising a single sgRNA targeting 50 target nucleic acids commonly comprised in the genomes of both Hela cells and HCT-116 cells, wherein the target sequences are the same.

FIG. 2 shows experimental results according to Experimental Example 2. Specifically, the results were obtained by treating HCT-116 cells with each of the substances listed in Table 1. Here, Olaparib (−) indicates a group not administered with a PARP inhibitor, and Olaparib (+) indicates a group administered with a PARP inhibitor. Additionally, Mock indicates a group administered with PBS only; NT indicates a group comprising a random sgRNA that does not target any sequence; Umix-4 indicates a group comprising sgRNAs targeting four target nucleic acids commonly comprised in the genomes of both Hela cells and HCT-116 cells; Hmix-4 indicates a group comprising sgRNAs targeting four target nucleic acids comprised only in the genome of HCT-116 cells; and MT-50 indicates a group comprising a single sgRNA targeting 50 target nucleic acids commonly comprised in the genomes of both Hela cells and HCT-116 cells, wherein the target sequences are the same.

FIG. 3 shows experimental results according to Experimental Example 3. Specifically, the results were obtained by treating Hela cells with each of the substances listed in Table 4. Here, Cas9^{WT} indicates a group administered with a ribonucleoprotein (RNP) comprising wild-type SpCas9 having double-strand break activity; nCas9^{D10A} indicates a group administered with an RNP comprising a nickase protein having single-strand break activity, which is a D10A mutant of SpCas9; and nCas9^{D10A}+Olaparib indicates a group administered with both an RNP comprising the nickase protein and olaparib, which is a PARP inhibitor. Additionally, Mock indicates a group administered with PBS only; NT indicates a group comprising a random sgRNA that does not target any sequence; Umix-4 indicates a group comprising sgRNAs targeting four target nucleic acids commonly comprised in the genomes of both Hela cells and HCT-116 cells; Hmix-4 indicates a group comprising sgRNAs targeting four target nucleic acids comprised only in the genome of HCT-116 cells; and MT-50 indicates a group comprising a single sgRNA targeting 50 target nucleic acids commonly comprised in the genomes of both Hela cells and HCT-116 cells, wherein the target sequences are the same.

FIG. 4 shows experimental results according to Experimental Example 3. Specifically, the results were obtained by treating HCT-116 cells with each of the substances listed in Table 4. Here, Cas9^{WT} indicates a group administered with an RNP comprising wild-type SpCas9 having double-strand break activity; nCas9^{D10A} indicates a group administered with an RNP comprising a nickase protein having single-strand break activity, which is a D10A mutant of SpCas9; and nCas9^{D10A}+Olaparib indicates a group administered with both an RNP comprising the nickase protein and olaparib, which is a PARP inhibitor. Additionally, Mock indicates a group administered with PBS only; NT indicates a group comprising a random sgRNA that does not target any sequence; Umix-4 indicates a group comprising sgRNAs targeting four target nucleic acids commonly comprised in the genomes of both HeLa cells and HCT-116 cells; Hmix-4 indicates a group comprising sgRNAs targeting four target nucleic acids comprised only in the genome of HCT-116 cells; and MT-50 indicates a group comprising a single sgRNA targeting 50 target nucleic acids commonly comprised in the genomes of both Hela cells and HCT-116 cells, wherein the target sequences are the same.

FIG. 5 shows experimental results according to Experimental Example 4. Specifically, the results were obtained by treating Hela cells with each of the substances listed in Table 5. Here, the x-axis of the graph represents the concentration of olaparib in μM, displayed on a logarithmic scale. Additionally, Non-treated indicates a group administered with a PARP inhibitor only; Umix-4 indicates a group comprising sgRNAs targeting four target nucleic acids commonly comprised in the genomes of both Hela cells and HCT-116 cells; Hmix-4 indicates a group comprising sgRNAs targeting four target nucleic acids comprised only in the genome of HCT-116 cells; and siBRCA2 indicates a group administered with an siRNA that silences the BRCA2 gene.

FIG. 6 shows experimental results according to Experimental Example 4. Specifically, the results were obtained by treating HCT-116 cells with each of the substances listed in Table 5. Here, the x-axis of the graph represents the concentration of olaparib in μM, displayed on a logarithmic scale. Additionally, Non-treated indicates a group administered with a PARP inhibitor only; Umix-4 indicates a group comprising sgRNAs targeting four target nucleic acids commonly comprised in the genomes of both Hela cells and HCT-116 cells; Hmix-4 indicates a group comprising sgRNAs targeting four target nucleic acids comprised only in the genome of HCT-116 cells; and siBRCA2 indicates a group administered with an siRNA that silences the BRCA2 gene.

FIG. 7 shows experimental results according to Experimental Example 5. Specifically, the results were obtained by treating Hela cells with each of the substances listed in Table 8. Here, Cas9^{WT} indicates a group administered with an RNP comprising wild-type SpCas9 having double-strand break activity; and nCas9^{D10A} indicates a group administered with an RNP comprising a nickase protein having single-strand break activity, which is a D10A mutant of SpCas9. Additionally, DMSO indicates a group administered with dimethyl sulfoxide (DMSO) without a PARP inhibitor; and Olaparib, Rucaparib, Niraparib, Veliparib, and Iniparib indicate groups administered with the corresponding PARP inhibitors, respectively. Furthermore, Mock indicates a group comprising PBS instead of an sgRNA; NT indicates a group comprising a random sgRNA that does not target any sequence; Umix-2 indicates a group comprising sgRNAs targeting two target nucleic acids commonly comprised in the genomes of both Hela cells and HCT-116 cells; Umix-3 indicates a group comprising sgRNAs targeting three target nucleic acids commonly comprised in the genomes of both Hela cells and HCT-116 cells; Umix-4 indicates a group comprising sgRNAs targeting four target nucleic acids commonly comprised in the genomes of both Hela cells and HCT-116 cells; and MT-50 indicates a group comprising a single sgRNA targeting 50 target nucleic acids commonly comprised in the genomes of both Hela cells and HCT-116 cells, wherein the target sequences are the same.

FIG. 8 shows experimental results according to Experimental Example 5. Specifically, the results were obtained by treating HCT-116 cells with each of the substances listed in Table 8. Here, Cas9^{WT} indicates a group administered with an RNP comprising wild-type SpCas9 having double-strand break activity; and nCas9^{D10A} indicates a group administered with an RNP comprising a nickase protein having single-strand break activity, which is a D10A mutant of SpCas9. Additionally, DMSO indicates a group administered with DMSO without a PARP inhibitor; and Olaparib, Rucaparib, Niraparib, Veliparib, and Iniparib indicate groups administered with the corresponding PARP inhibitors, respectively. Furthermore, Mock indicates a group comprising PBS instead of an sgRNA; NT indicates a group comprising a random sgRNA that does not target any sequence; Umix-2 indicates a group comprising sgRNAs targeting two target nucleic acids commonly comprised in the genomes of both Hela cells and HCT-116 cells; Umix-3 indicates a group comprising sgRNAs targeting three target nucleic acids commonly comprised in the genomes of both Hela cells and HCT-116 cells; Umix-4 indicates a group comprising sgRNAs targeting four target nucleic acids commonly comprised in the genomes of both Hela cells and HCT-116 cells; and MT-50 indicates a group comprising a single sgRNA targeting 50 target nucleic acids commonly comprised in the genomes of both Hela cells and HCT-116 cells, wherein the target sequences are the same.

FIG. 9 shows experimental results according to Experimental Example 6. Specifically, the results show mutation efficiencies measured as a result of treating Hela cells and HCT-116 cells, respectively, with RNPs targeting the HPRT1, BCR, DNAJC6, and PLXDC2 genes are shown. Here, Cas9^{WT} indicates a group administered with an RNP comprising wild-type SpCas9 having double-strand break activity; nCas9^{D10A} indicates a group administered with an RNP comprising a nickase protein having single-strand break activity, which is a D10A mutant of SpCas9; and nCas9^{D10A}+Olaparib indicates a group administered with both an RNP comprising the nickase protein and olaparib, which is a PARP inhibitor.

FIG. 10 is a schematic diagram illustrating the reasons why mutation frequencies differ depending on the type of Cas protein in Experimental Example 6.

FIG. 11 shows experimental results according to Experimental Example 7. Specifically, the results were obtained by administering substances listed in Table 14 to a mouse xenograft model implanted with HCT-116 tumors, and then measuring the tumor volume daily. The tumor volume was measured in units of mm3 according to Experimental Example 1.11. Here, PBS indicates the tumor volume of mice administered with PBS only; Olaparib only indicates the tumor volume of mice administered with olaparib only; RNP (Hmix-4) only indicates the tumor volume of mice administered with only an RNP comprising an nCas protein and the Hmix-4 guide RNA listed in Table 14; and RNP (Hmix-4)+Olaparib indicates the tumor volume of mice administered with both olaparib and the RNP.

FIG. 12 shows experimental results according to Experimental Example 7. Specifically, the results were obtained by subjecting tumor sections from mice administered with each substance in Experimental Example 7 to Ki-67, Caspase-3, and TUNEL assays according to Experimental Examples 1.13 and 1.14. Here, PBS indicates the tumor of mice administered with PBS only; Olaparib indicates the tumor of mice administered with olaparib only; Hmix-4 indicates the tumor of mice administered with only an RNP comprising an nCas protein and the Hmix-4 guide RNA listed in Table 14; and Hmix-4+Olaparib indicates the tumor of mice administered with both olaparib and the RNP.

FIG. 13 shows experimental results according to Experimental Example 7. Specifically, images of tumor tissues from mice sacrificed on day 25 are shown. Here, PBS indicates the tumor of mice administered with PBS only; Olaparib indicates the tumor of mice administered with olaparib only; Hmix-4 indicates the tumor of mice administered with only an RNP comprising an nCas protein and the Hmix-4 guide RNA listed in Table 14; and Hmix-4+Olaparib indicates the tumor of mice administered with both olaparib and the RNP.

### DETAILED DESCRIPTION

Hereinafter, the best mode for carrying out the invention is exemplarily disclosed. This includes some embodiments of the invention disclosed in the present specification, but does not include all possible embodiments. The embodiments described in this paragraph are merely illustrative, and the embodiments described herein should not be understood as the only “best mode” of the present invention. A person having ordinary skill in the art may conceive of many modifications and more preferable embodiments based on the examples described in this paragraph, and such modifications and embodiments should also be considered as being included in the best mode for carrying out the present invention.

This specification discloses a method for killing a target cell, wherein the target cell comprises three or more target nucleic acids in its genome,
the method comprises the following:
delivering a CRISPR/nCas composition for causing targeted cell death, and a PARP inhibitor to the target cell,
wherein, the CRISPR/nCas composition for causing targeted cell death causes single-strand breaks at three or more target nucleic acids in the genome of the target cell,
the CRISPR/nCas composition for causing targeted cell death and the PARP inhibitor act together to induce cell death,
wherein, the CRISPR/nCas composition for causing targeted cell death comprises one or more types of CRISPR/nCas compositions,
wherein each type of CRISPR/nCas composition comprises:
a Cas protein having single-strand break activity, or a nucleic acid encoding the Cas protein; and
a guide RNA comprising a guide domain and a scaffold, or a nucleic acid encoding the guide RNA;
wherein, the scaffold interacts with the Cas protein such that the guide RNA forms a complex with the Cas protein, and
the guide domain of the guide RNA targets one of the nucleic acid sequences of the target nucleic acids.

In one embodiment,
the target cell comprises at least three target nucleic acids having different nucleic acid sequences from each other, and
wherein the CRISPR/nCas composition for causing targeted cell death comprises at least three types of CRISPR/nCas compositions,
the guide domain of the guide RNA of each type of CRISPR/nCas composition targets different nucleic acid sequence from each other,
the guide domain of the guide RNA of each type of CRISPR/nCas composition targets one of the nucleic acid sequences of the target nucleic acids.

In one embodiment,
the target cell comprises at least three target nucleic acids having the identical nucleic acid sequence,
the CRISPR/nCas composition for causing targeted cell death comprises at least one type of CRISPR/nCas compositions, and
the guide domain of the guide RNA of each type of CRISPR/nCas composition targets the nucleic acid sequence of the target nucleic acids.

In one embodiment, each type of CRISPR/nCas composition is in a form selected from:
a composition comprising the Cas protein having single-strand break activity and the guide RNA, wherein the Cas protein binds to the guide RNA to form an RNA-protein complex; or
a composition comprising an mRNA encoding the Cas protein having single-strand break activity, and the guide RNA.

In one embodiment,
the PARP inhibitor is selected from i) Olaparib; ii) Rucaparib; iii) Niraparib; iv) Veliparib; v) Iniparib; or combination of any of i) to v).

In one embodiment,
the target nucleic acids are double-stranded DNA comprising a target strand and a non-target strand, and
the targeting one of the nucleic acid sequences of the target nucleic acids by the guide domain of the guide RNA of the CRISPR/nCas composition, is selected from:
a guide domain having an RNA sequence equivalent to one of DNA sequences of the non-target strand of the target nucleic acids;
a guide domain being capable of hybridizing with one of DNA sequences of the target strand of the target nucleic acids; or
a guide domain having an RNA sequence complementary to one of DNA sequences of the target strand of the target nucleic acids.

This specification discloses a method for treating a cancer by selectively killing cancer cells of a patient,
the cancer cell of the patient comprises three or more target nucleic acids which are not present in a normal cell,
the method of treating the cancer comprises:
administering a CRISPR/nCas composition for causing targeted cell death, and a PARP inhibitor to the patient,
wherein, the CRISPR/nCas composition for causing targeted cell death causes single-strand breaks at three or more target nucleic acids in the genome of the cancer cell of the patient,
the CRISPR/nCas composition for causing targeted cell death and the PARP inhibitor act together to induce the cancer cell death,
wherein, the CRISPR/nCas composition for causing targeted cell death comprises one or more types of CRISPR/nCas compositions,
each type of CRISPR/nCas composition comprises:
a Cas protein having single-strand break activity, or a nucleic acid encoding the Cas protein; and
a guide RNA comprising a guide domain and a scaffold, or a nucleic acid encoding the guide RNA;
wherein, the scaffold interacts with the Cas protein such that the guide RNA forms a complex with the Cas protein, and
the guide domain of the guide RNA targets one of the nucleic acid sequences of the target nucleic acids.

In one embodiment,
the cancer cell comprises at least three target nucleic acids having different nucleic acid sequences from each other,
the CRISPR/nCas composition for causing targeted cell death comprises at least three types of CRISPR/nCas compositions,
the guide domain of the guide RNA of each type of CRISPR/nCas composition targets different nucleic acid sequence from each other, and
the guide domain of the guide RNA of each type of CRISPR/nCas composition targets one of the nucleic acid sequences of the target nucleic acids.

In one embodiment,
the cancer cell comprises at least three target nucleic acids having the same nucleic acid sequence,
the CRISPR/nCas composition for causing targeted cell death comprises at least one type of CRISPR/nCas compositions, and
the guide domain of the guide RNA of each type of CRISPR/nCas composition targets the nucleic acid sequence of the target nucleic acids.

In one embodiment,
the PARP inhibitor is selected from i) Olaparib; ii) Rucaparib; iii) Niraparib; iv) Veliparib; v) Iniparib; or combination of any of i) to v).

In one embodiment,
the CRISPR/nCas composition for causing targeted cell death and the PARP inhibitor are each independently administered to the patient.

In one embodiment,
each type of CRISPR/nCas composition is in a form selected from:
a composition comprising the Cas protein having single-strand break activity and the guide RNA, wherein the Cas protein binds to the guide RNA to form an RNA-protein complex; or
a composition comprising an mRNA encoding the Cas protein having single-strand break activity, and the guide RNA.

In one embodiment,
the target nucleic acids are double-stranded DNA comprising a target strand and a non-target strand, and
the targeting one of the nucleic acid sequences of the target nucleic acids by the guide domain of the guide RNA of the CRISPR/nCas composition is selected from:
a guide domain having an RNA sequence equivalent to one of DNA sequences of the non-target strand of the target nucleic acids;
a guide domain being capable of hybridizing with one of DNA sequences of the target strand of the target nucleic acids; or
a guide domain having an RNA sequence complementary to one of DNA sequences of the target strand of the target nucleic acids.

This specification discloses a method of killing a target cell, wherein the target cell comprises a first target nucleic acid, a second target nucleic acid, and a third target nucleic acid in its genome,
wherein the method comprises:
delivering a CRISPR/nCas composition for causing targeted cell death, and a PARP inhibitor to the target cell,
wherein, the CRISPR/nCas composition for causing targeted cell death comprises:
a first CRISPR/nCas complex comprising a first nCas protein, and a first guide RNA;
a second CRISPR/nCas complex comprising a second nCas protein, and a second guide RNA; and
a third CRISPR/nCas complex comprising a third nCas protein, and a third guide RNA;
wherein, the first CRISPR/nCas complex causes single-strand breaks at the first target nucleic acid,
the second CRISPR/nCas complex causes single-strand breaks at the second target nucleic acid,
the third CRISPR/nCas complex causes single-strand breaks at the third target nucleic acid, and
the CRISPR/nCas composition and the PARP inhibitor act together to induce the target cell death.

This specification discloses a method for killing a target cell, wherein the target cell comprises a first target nucleic acid, a second target nucleic acid, and a third target nucleic acid in its genome,
wherein the method comprises:
delivering a CRISPR/nCas composition for causing targeted cell death, and a PARP inhibitor to the target cell,
wherein, the CRISPR/nCas composition for causing targeted cell death comprises:
a first CRISPR/nCas composition comprising an mRNA encoding a first nCas protein, and a first guide RNA;
a second CRISPR/nCas composition comprising an mRNA encoding a second nCas protein, and a second guide RNA; and
a third CRISPR/nCas composition comprising an mRNA encoding a third nCas protein, and a third guide RNA;
wherein, the first CRISPR/nCas composition induces formation of a first CRISPR/nCas complex, in which the first nCas protein and the first guide RNA are combined, within the target cell,
the second CRISPR/nCas composition induces a formation of a second CRISPR/nCas complex in which the second nCas protein and the second guide RNA are combined within the target cell, and
the third CRISPR/nCas composition induces a formation of a third CRISPR/nCas complex in which the third nCas protein and the third guide RNA are combined within the target cell,
wherein, the first CRISPR/nCas complex causes single-strand breaks at the first target nucleic acid,
the second CRISPR/nCas complex causes single-strand breaks at the second target nucleic acid,
the third CRISPR/nCas complex causes single-strand breaks at the third target nucleic acid, and
the CRISPR/nCas composition and the PARP inhibitor act together to induce the target cell death.

In one embodiment,
the target cell is a cancer cell, and the first target nucleic acid, the second target nucleic acid, and the third target nucleic acid are cancer cell-specific target nucleic acids.

### MODE FOR INVENTION

Hereinafter, the content of the present disclosure will be described in more detail through specific embodiments and examples with reference to the attached drawings. It should be noted that the attached drawings include some, but not all, embodiments of the present disclosure. The content of the present disclosure disclosed herein can be embodied in many different forms and is not limited to the specific embodiments described herein. Such embodiments should be viewed as provided to satisfy the legal requirements applied herein. Those skilled in the art to which the present disclosure pertains will readily appreciate that various modifications and other embodiments regarding the content of the present disclosure are possible. Therefore, it should be understood that the content of the present disclosure is not limited to the specific embodiments described herein, and modifications and other embodiments thereof also fall within the scope of the claims.

### Definition of Terms

### About

As used herein, the term “about” refers to an amount, level, value, number, frequency, percent, dimension, size, amount, weight, or length that varies by 30%, 25%, 20%, 15%, 10%, 9%, 8%, 7%, 6%, 5%, 4%, 3%, 2%, 1%, or 0% with respect to a reference amount, level, value, number, frequency, percent, dimension, size, amount, weight, or length.

### Amino Acid Sequence Notation

Unless otherwise stated, amino acid sequences are described herein in the N-terminal to C-terminal direction, using either a one-letter or three-letter notation of amino acids. For example, when written as MDK, this denotes a peptide in which methionine, aspartic acid, and lysine are linked in this order in the N-terminal to C-terminal direction. In another example, when written as Lys-Tyr-Ser, this denotes a peptide in which lysine, tyrosine, and serine are linked in this order in the N-terminal to C-terminal direction. Amino acids that cannot be denoted by the one-letter notation may be notated using other letters and further elaborated.

Each amino acid is notated in the following manner: alanine (Ala, A); arginine (Arg, R); asparagine (Asn, N); aspartic acid (Asp, D); cysteine (Cys, C); glutamic acid (Glu, E); glutamine (Gln, Q); glycine (Gly, G); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V).

### Nucleic Acid Sequence Notation

As used herein, the symbols A, T, C, G, and U are to be interpreted as having the meanings commonly understood by those skilled in the art, and may be interpreted appropriately as a base, nucleoside, or nucleotide in DNA or RNA, depending on the context and technology. For example, the respective symbols may be interpreted as adenine (A), thymine (T), cytosine (C), guanine (G), or uracil (U) when referring to a base, or as adenosine (A), thymidine (T), cytidine (C), guanosine (G), or uridine (U) when referring to a nucleoside. Additionally, when a symbol refers to a nucleotide in a sequence, it should be interpreted as a nucleotide comprising the corresponding nucleoside described above. When referring to or notating a nucleic acid sequence, the sequence is described in the direction from the 5′ end to the 3′ end of the nucleic acid.

### Background Art—CRISPR/Cas System

### Overview of CRISPR/Cas System

The CRISPR/Cas system, which is a type of immune system found in prokaryotic organisms, comprises a Cas protein and a guide RNA. Detailed configurations of the Cas protein or the guide RNA are described in the published document WO 2018/231018 (International Publication Number). As used herein, the term “Cas protein” collectively refers to nucleases that may be interpreted as being used in the CRISPR/Cas system. Hereinafter, the DNA cleavage process of the most commonly used CRISPR/Cas9 system will be briefly described.

### Cas9 Protein

In a CRISPR/Cas9 complex, a protein having nuclease activity that cleaves a nucleic acid is referred to as a Cas9 protein. The Cas9 protein corresponds to Class 2, Type II in the CRISPR/Cas system classification, and may be derived from Streptococcus pyogenes, Streptococcus thermophilus, Streptococcus sp., Streptomyces pristinaespiralis, Streptomyces viridochromogenes, Streptomyces viridochromogenes, Streptosporangium roseum, or Streptosporangium roseum.

### Guide RNA

In a CRISPR/Cas9 complex, an RNA having a function of guiding the CRISPR/Cas9 complex to recognize a specific sequence comprised in a target nucleic acid is referred to as a guide RNA. The configuration of the guide RNA may be broadly divided into a scaffold and a guide domain. The scaffold is a portion that interacts with a Cas9 protein and enables binding to the Cas9 protein to form a complex. In general, the scaffold comprises a tracrRNA and a repeat sequence region of a crRNA. Specific nucleic acid sequences may vary depending on the type of Cas9 protein used in combination. The guide domain is a portion capable of complementary binding to all or a part of the target nucleic acid. The guide domain is designed artificially depending on the target nucleic acid.

### Target Nucleic Acid Cleavage Process by CRISPR/Cas9 Complex

When a CRISPR/Cas9 complex comes into contact with a target nucleic acid such that a Cas9 protein recognizes a nucleotide sequence of a predetermined length, and a guide domain of a guide RNA complementarily binds to a nucleic acid adjacent to the sequence recognized by the Cas9 protein, the target nucleic acid is cleaved by the CRISPR/Cas9 complex. In this case, the nucleotide sequence of a predetermined length, recognized by the Cas9 protein, is referred to as a protospacer-adjacent motif (PAM). The PAM is a sequence determined by the type or origin of the Cas9 protein. For example, the Cas9 protein derived from Streptococcus pyogenes can recognize the 5′-NGG-3′ sequence within the target nucleic acid, wherein N represents one of A, T, C, or G. In order for the CRISPR/Cas9 complex to cleave the target nucleic acid, the guide domain of the guide RNA must bind complementarily to the sequence portion adjacent to the PAM. Here, when the target nucleic acid is double-stranded DNA, the guide RNA binds complementarily to the strand that does not comprise the PAM. This will be described in more detail in the section below entitled “Target Strand and Non-Target Strand”. Therefore, the guide sequence portion is designed to match the sequence of the target nucleic acid, specifically the sequence portion adjacent to the PAM sequence. When the CRISPR/Cas9 complex cleaves the target nucleic acid, any site within the PAM sequence portion of the target nucleic acid and/or the sequence portion that binds complementarily to the guide domain will be cleaved.

### Target Strand and Non-Target Strand

The CRISPR/Cas9 complex has break activity with respect to double-stranded DNA. In double-stranded DNA, a strand that binds to the guide domain is referred to as a target strand (TS). A strand complementary to the target strand, which does not bind to the guide domain and comprises a PAM recognized by the Cas9 protein and a protospacer, is referred to as a non-target strand (NTS). The guide domain has a nucleic acid sequence equivalent to the protospacer of the NTS. The guide domain has a nucleic acid sequence complementary to a predetermined portion of the TS.

### Background Art—CRISPR/Cas System Having Single-Strand Break Activity

The CRISPR/Cas system, commonly used for cell genome editing, cleaves double-stranded nucleic acids in a target-specific manner. However, in some cases, it is necessary to cleave only a single strand rather than the entire double-stranded nucleic acid. The CRISPR/Cas system used for this purpose is a CRISPR/Cas system having single-strand break activity. This CRISPR/Cas system, having single-strand break activity is also referred to as a nickase or CRISPR/nCas system. This nickase recognizes double-stranded nucleic acids (for example, double-stranded DNA) in a target-specific manner but cleaves only a single strand of the double-stranded nucleic acid. The CRISPR/nCas system is typically produced by artificially modifying certain domains of the Cas protein having double-strand break activity. For example, it is known that when histidine at position 840 in the amino acid sequence of a Cas9 protein derived from Streptococcus pyogenes is substituted with alanine (H840A mutation), or when aspartic acid at position 10 is substituted with alanine (D10A mutation), one of the domains of the Cas9 protein responsible for cleaving each nucleic acid strand is inactivated, such that the Cas9 protein can be utilized as a nickase.

### Background Art—PARP

### Overview of PARP

PARP, which is a family of proteins that plays a key role in the DNA damage response and DNA repair, is mainly present in the cell nucleus. In humans, 17 PARP proteins have been identified, among which PARP1 and PARP2 play the most important roles in DNA repair. PARP catalyzes poly(ADP-ribosyl) ation, a process in which ADP-ribose polymers are attached to target proteins using NAD+ as a substrate. This process affects various cellular functions and is involved particularly in DNA repair, transcriptional regulation, cell death, chromatin remodeling, and the like. As described above, PARP plays a key role in maintaining genome stability. These diverse functions of PARP are closely associated with pathological processes of various diseases, including cancer, inflammatory diseases, and neurodegenerative diseases. Thus, PARP is an important research subject.

### Key Function of PARP—PARP-Mediated DNA Repair

PARP detects the occurrence of single-strand breaks (SSBs) in DNA and facilitates single-strand break repair. When a single-strand DNA break occurs, PARP binds to the SSB site and is activated. For example, PARP1 binds to the damage site via an N-terminal zinc finger domain, whereas PARP2 binds to the damage site via a different specific domain. Activated PARP synthesizes PAR chains using NAD+ as a substrate. The synthesized PAR chains recruit repair proteins, including XRCC1, DNA ligase III, DNA polymerase beta, and kinases, to the damage site. The SSB site is repaired by these various enzymes, and such a process is referred to as base excision repair (BER).

### Therapeutic Uses of PARP Inhibitors and Limitations Thereof

When a PARP inhibitor is administered, PARP-mediated DNA repair is inhibited in cells. Administration of a PARP inhibitor to carcinomas such as ovarian cancer and breast cancer, in which a homologous recombination repair (HRR) mechanism does not function properly due to the occurrence of mutations in BRCA1 and BRCA2, can further inhibit single-strand DNA repair capacity, resulting in the accumulation of genomic damage and, ultimately, cancer cell death. This phenomenon is referred to as synthetic lethality. Currently, PARP inhibitors approved by the U.S. Food and Drug Administration (FDA) include olaparib, rucaparib, and niraparib. These PARP inhibitors are used alone or in combination with other anticancer agents. However, PARP inhibitors have the following limitations: 1) limited efficacy in carcinomas without BRCA mutations; 2) development of resistance in cancer cells upon long-term administration; and 3) serious side effects such as bone marrow suppression, fatigue, vomiting, anemia, and thrombocytopenia when administered at doses sufficient to induce cancer cell death.

### Method for Killing Target Cell Using CRISPR/nCas Composition for Cell Death and PARP Inhibitor

### Overview of Method for Killing Target Cell

In this specification, a method for killing a target cell using a CRISPR/nCas composition for cell death and a PARP inhibitor is disclosed. Specifically, this method comprises a process of delivering a CRISPR/nCas composition for cell death and a PARP inhibitor to a target cell. The CRISPR/nCas composition for cell death induces single-strand breaks at three or more target nucleic acids within the genome of the target cell. The PARP inhibitor inhibits the action of PARP as described above. The CRISPR/nCas composition for cell death and the PARP inhibitor act together within the target cell to induce targeted cell death. The inventors of this specification presume that, when the CRISPR/nCas composition for cell death and the PARP inhibitor act together, double-strand breaks occur at three or more nucleic acids within the genome of cells, thereby inducing cell death.

### Key Concepts for Explaining Method for Killing Target Cell

(i) The target cell has three or more target nucleic acids within its genome. The respective target nucleic acids may have the same nucleic acid sequence or may have different nucleic acid sequences.
(ii) The CRISPR/nCas composition for cell death comprises one or more units of a CRISPR/nCas system. A single unit of a CRISPR/nCas system recognizes one type of nucleic acid sequence and induces a single-strand break. The CRISPR/nCas composition for cell death comprises a sufficient number of units of a CRISPR/nCas system to recognize the three or more target nucleic acids and induce single-strand breaks.
(iii) When the CRISPR/nCas composition for cell death is delivered to the target cell, single-strand breaks occur at three or more target nucleic acids within the genome.
(iv) The PARP inhibitor inhibits the function of PARP within the cell.
(v) When the PARP inhibitor is delivered to the target cell, PARP-mediated single-stranded nucleic acid repair mechanisms in the target cell are inhibited.
(vi) The target cell to which both the CRISPR/nCas composition for cell death and the PARP inhibitor are delivered undergoes cell death.

Hereinafter, the key concepts will be described in more detail.

### Target Cell

In the method for killing the target cell disclosed herein, the target cell refers to a cell subject to cell death. In the method for killing the target cell, the type of target cell subject to cell death is not particularly limited. The target cell has three or more target nucleic acids within its genome. The target nucleic acid will be described in more detail in the sections below. In other words, the target cell is selected appropriately depending on the purpose and use of the above cell death method. For example, the target cell may be a cancer cell or senescent cell.

### Target Nucleic Acid

A target nucleic acid refers to a nucleic acid region having a specific sequence at a specific site. In this specification, the target nucleic acid is used as a concept encompassing both a nucleic acid sequence and its site. For example, a target nucleic acid comprised in a specific subject may be identified as a nucleic acid region that (i) belongs to a specific subject, (ii) starts from a base at a specific site, and (iii) ends at a base at a specific site. Here, instead of a base, a nucleoside or a nucleotide may be identified, and such terms are to be interpreted appropriately according to the context. The specific subject may be, for example, a genome of a specific cell, a specific chromosome, a specific gene, or a specific genetic region, and may be any subject for which a site and a sequence can be identified. A sequence in which the types of bases continuously arranged from the starting base to the ending base are listed is referred to as the sequence of the target nucleic acid. The number of continuously arranged bases from the starting base to the ending base is referred to as the length of the target nucleic acid. The target nucleic acid may refer to a double-stranded or single-stranded nucleic acid, depending on the context in which the target nucleic acid is defined. Even when the target nucleic acid refers to a double-stranded nucleic acid, the description is made on the basis of one strand for convenience. However, it should be understood that this target nucleic acid comprises another strand complementary to the strand described above.

Target Cell Having Three or More Target Nucleic Acids within Genome

The fact that a target cell has three or more target nucleic acids within its genome means that three or more different target nucleic acids can be defined within the genome of the target cell. In order to use the method for killing the target cell described above, at least three target nucleic acids must be identified within the genome of the target cell. As described above, the sequences of the respective target nucleic acids may be the same, but may have different sites.

### CRISPR/nCas Composition for Cell Death

The method for killing the target cell uses a CRISPR/nCas composition for cell death. When the CRISPR/nCas composition for cell death is delivered to the target cell, single-strand breaks occur at three or more target nucleic acids within the genome of the target cell. The CRISPR/nCas composition for cell death comprises one or more units of a CRISPR/nCas system. The single unit of a CRISPR/nCas system targets one type of nucleic acid sequence and induces a single-strand break. The CRISPR/nCas composition for cell death comprises a sufficient number of units of a CRISPR/nCas system to recognize the three or more target nucleic acids and induce single-strand breaks. The CRISPR/nCas composition for cell death can be implemented in various ways. Specific details will be described in the section entitled “CRISPR/nCas Composition for Cell Death” and in the section entitled “Possible Embodiments of Present Disclosure”, specifically in the subsection entitled “CRISPR/nCas Composition for Cell Death”.

### PARP Inhibitor

The method for killing the target cell uses a PARP inhibitor. The PARP inhibitor inhibits the function of PARP described above, especially the single-strand break repair function. The PARP inhibitor prevents PARP-mediated single-strand repair from occurring when the CRISPR/nCas composition for cell death induces single-strand breaks within the genome of the target cell. As used herein, the PARP inhibitor is not limited in form, composition, or type, as long as it inhibits the single-strand break repair function of PARP. For example, the PARP inhibitor may be olaparib, rucaparib, niraparib, veliparib, or iniparib.

### Delivering CRISPR/nCas Composition for Cell Death and PARP Inhibitor to Target Cell

To induce targeted cell death, the CRISPR/nCas composition for cell death and the PARP inhibitor must be delivered to the target cell together. As long as the above purpose is achieved, the method for delivering the CRISPR/nCas composition for cell death and the PARP inhibitor to the target cell is not particularly limited. The CRISPR/nCas composition for cell death and the PARP inhibitor may be delivered to the target cell in combination or independently of each other, using known methods.

### Killing Cells by Combined Action of CRISPR/nCas Composition for Cell Death and PARP Inhibitor

Experimental results show that only target cells to which both the CRISPR/nCas composition for cell death and the PARP inhibitor are delivered together undergo cell death. Accordingly, it can be concluded that the CRISPR/nCas composition for cell death and the PARP inhibitor act together to induce cell death.

The inventors of this specification believe that cell death is induced according to the following principles.
(a) When the CRISPR/nCas composition for cell death is delivered to a target cell, single-strand breaks occur at three or more nucleic acids within the genome of the target cell.
(b) When the PARP inhibitor is delivered to the target cell, PARP-mediated single-stranded nucleic acid repair mechanisms in the target cell are inhibited.
(c) Therefore, when both the CRISPR/nCas composition for cell death and the PARP inhibitor are delivered together to the target cell, single-strand breaks occur at three or more nucleic acids within the genome of the target cell. However, the broken nucleic acids are not properly repaired due to inhibition of the PARP-mediated single-stranded nucleic acid repair mechanisms.
(d) It is known that, when single-strand breaks occur, unless the PARP-mediated single-stranded nucleic acid repair mechanisms function, the single-strand breaks develop into double-strand breaks (reference).
(e) Consequently, when both the CRISPR/nCas composition for cell death and the PARP inhibitor are delivered together to the target cell, double-strand breaks occur at three or more nucleic acids within the genome of the cell, thereby inducing cell death.

### Advantage #1 of Cell Death Method—Excellent Effect of Inducing Targeted Cell Death

By using the cell death method disclosed herein, targeted cell death can be efficiently induced. In particular, cell death can be induced with an efficiency equivalent to or greater than that in conventional techniques for inducing targeted cell death using CRISPR/Cas systems having double-strand break activity.

### Advantage #2 of Cell Death Method—Selective Induction of Targeted Cell Death

The cell death method disclosed herein selectively induces targeted cell death. The inventors of this specification have demonstrated through experiments that the cell death method selectively induces only targeted cell death without causing death of non-target cells. The cell death method does not exhibit undesired cytotoxicity to non-target cells, which constitutes a significant advantage when this technique is applied to therapeutic agents.

### Advantage #3 of Cell Death Method—No Undesired Effects on Non-Target Cells

When the above-described cell death method is used, cases in which non-target cells may be affected include the following:
(i) the CRISPR/nCas composition for cell death is delivered to non-target cells; (ii) the PARP inhibitor is delivered to non-target cells; or (iii) both the CRISPR/nCas composition for cell death and the PARP inhibitor are delivered together to non-target cells.

In case (i), non-target cells do not comprise the target nucleic acid and are therefore unaffected by the CRISPR/nCas composition for cell death. Even when the CRISPR/nCas composition for cell death was to function due to misrecognition of non-target nucleic acids, the fact that CRISPR/Cas systems having single-strand break activity are less likely to introduce undesired mutations into the genome of the cells is well known.

In case (ii), the effects of the PARP inhibitor on non-target cells may be problematic. However, the inventors of this specification have found that, even when the PARP inhibitor is used at a dose sufficiently low so as not to affect ordinary cells, a cell death effect can be induced when the PARP inhibitor is used together with the CRISPR/nCas composition for cell death. Accordingly, according to the above-described cell death method, no significant side effects occur even when the PARP inhibitor is delivered to non-target cells.

In case (iii), similarly to case (i), the probability that the CRISPR/nCas composition for cell death causes problems in non-target cells is low. Furthermore, the inventors of this specification have found that, when both the PARP inhibitor and the CRISPR/nCas system are introduced together, the frequency of introducing mutations (that is, indels) into the target nucleic acid is even lower than that when the CRISPR/nCas system is introduced alone. As a result, even when the CRISPR/nCas composition for cell death functions due to misrecognition of a non-target nucleic acid, the probability that undesired mutations are introduced into the genome of non-target cells would be significantly low.

In case (iii), problems may arise when the CRISPR/nCas composition for cell death functions due to misrecognition of a non-target nucleic acid, and acts together with the PARP inhibitor to induce development into double-strand breaks. However, the probability that the CRISPR/nCas composition functions due to misrecognition of a non-target nucleic acid is itself not high. Additionally, because cell death occurs only when double-strand breaks occur in a number equal to or greater than a predetermined threshold, the probability that non-target cells are killed due to misrecognition of a non-target nucleic acid by the CRISPR/nCas composition is negligible. In fact, the inventors have demonstrated through experiments that non-target cells are not killed even when both the CRISPR/nCas composition and the PARP inhibitor are delivered together to non-target cells.

In summary, in any of the above cases (i) to (iii), the probability that either or both of the CRISPR/nCas composition and the PARP inhibitor introduced into non-target cells induce cytotoxicity or introduce undesired mutations into the genome of non-target cells is significantly low.

### CRISPR/nCas Composition for Cell Death

### Overview of CRISPR/nCas Composition for Cell Death

The method for killing the target cell disclosed herein uses a CRISPR/nCas composition for cell death. The CRISPR/nCas composition for cell death may induce single-strand breaks at three or more nucleic acids within the genome of the target cell. The CRISPR/nCas composition for cell death comprises one or more units of a CRISPR/nCas system. Depending on the target nucleic acid identified in the genome of the target cell, the CRISPR/nCas composition for cell death can be implemented in various ways. To explain this, a single unit of a CRISPR/nCas system will first be described, followed by an explanation of how the CRISPR/nCas composition for cell death is implemented using the same.

### Overview of Single Unit of CRISPR/nCas System

A single unit of a CRISPR/nCas system conceptually refers to a CRISPR/Cas system capable of inducing a single-strand break by targeting one type of nucleic acid sequence. This CRISPR/nCas system encompasses not only an RNA-protein complex that directly acts on a nucleic acid to cleave a single-strand nucleic acid, but also all various implementation forms that enable the RNA-protein complex to be expressed within a cell.

### Single Unit of CRISPR/nCas System—Definition of Single Unit

A single unit of a CRISPR/nCas system refers to a CRISPR/Cas system capable of inducing a single-strand break by targeting one type of nucleic acid sequence. The single unit of a CRISPR/nCas system comprises one type of guide RNA and one type of Cas protein having single-strand break activity. The term “single unit” is used to distinguish such a system from cases in which a plurality of guide RNAs or a plurality of Cas proteins are referred to. For example, the presence of 100 guide RNA molecules and 50 Cas protein molecules having single-strand break activity is still regarded as a single unit of a CRISPR/nCas system when all of the guide RNA sequences are the same, and all of the Cas protein sequences are the same.

### Component #1 of Single Unit of CRISPR/nCas System—Cas Protein Having Single-Strand Break Activity

The single unit of a CRISPR/nCas system comprises one type of Cas protein having single-strand break activity. The Cas protein having single-strand break activity is also referred to as a nickase or an nCas protein. The Cas protein having single-strand break activity has been described in the section above entitled “Background Art-CRISPR/Cas System Having Single-Strand Break Activity”.

### Component #2 of Single Unit of CRISPR/nCas System—Guide RNA

The single unit of a CRISPR/nCas system comprises one type of guide RNA. The guide RNA comprises a scaffold that interacts with the nCas protein to form a complex, and a guide domain capable of recognizing a specific nucleic acid sequence. The linkage structure between the scaffold and the guide domain varies depending on the type of nCas protein capable of forming a complex with the guide RNA. For example, when the nCas protein is a Cas9 protein or a variant thereof, the guide RNA has a structure in which the guide domain and the scaffold are sequentially linked in the direction from the 5′ end to the 3′ end. In another example, when the nCas protein is a Cas12a protein or a variant thereof, the guide RNA has a structure in which the scaffold and the guide domain are sequentially linked in the direction from the 5′ end to the 3′ end.

### Guide RNA #1—Scaffold

The guide RNA comprises a scaffold. The scaffold enables the guide RNA to interact with the nCas protein to form a complex. The nucleic acid sequence of the scaffold of the guide RNA that interacts with the nCas protein varies depending on the type of nCas protein. Since the guide RNA of a CRISPR/Cas system is well known, those skilled in the art would readily understand which portion is referred to as the scaffold herein. For example, when the nCas protein is a Cas9 protein or a variant thereof, the scaffold comprises a tracrRNA and a direct repeat region of a crRNA. In another example, when the nCas protein is a Cas12a protein or a variant thereof, the scaffold comprises a direct repeat region of a crRNA. In some cases, the scaffold may consist of a single molecule, or may encompass two or more molecules.

### Guide RNA #2—Guide Domain

The guide RNA comprises a guide domain. The guide domain is designed to recognize a specific nucleic acid sequence. Specifically, the guide domain is designed to enable complementary binding to all or a part of the target nucleic acid. When the nCas protein recognizes a PAM sequence and the guide domain of the guide RNA recognizes a nucleic acid sequence adjacent to the PAM sequence, the CRISPR/nCas system functions to induce single-strand breaks. Various embodiments of the guide domains are described in the section below entitled “Guide Domain” under “Possible Embodiments of Present Disclosure”.

### Guide RNA #3—Other Domains

The guide RNA may comprise one or more other domains in addition to the scaffold and guide domain. These other domains are adopted for different purposes and arranged in an appropriate site. For example, this other domain may be a guide RNA stabilization motif.

### Form #1 of Single Unit of CRISPR/nCas System—Protein-RNA Complex

The single unit of a CRISPR/nCas system may refer to a protein-RNA complex in which the nCas protein and the guide RNA form a complex. The protein-RNA complex may also be referred to as a CRISPR/nCas complex or an RNP. The protein-RNA complex comes into contact with the target nucleic acid to induce single-strand breaks.

### Form #2 of Single Unit of CRISPR/nCas System—Vector or Encoding Nucleic Acid

The single unit of a CRISPR/nCas system may refer to a vector capable of expressing the nCas protein and the guide RNA, respectively, or to nucleic acids encoding the same. The vector or the encoding nucleic acids may be collectively referred to as a CRISPR/nCas vector. The CRISPR/nCas vector is configured such that, when introduced into a target cell, the nCas protein and the guide RNA are each expressed.

### Form #3 of Single Unit of CRISPR/nCas System—Composition

The single unit of a CRISPR/nCas system may refer to a composition comprising: an nCas protein or a nucleic acid encoding the same; and a guide RNA or a nucleic acid encoding the same. The composition may be referred to as a CRISPR/nCas composition. When the CRISPR/nCas composition is introduced into a target cell, the genome of the target cell may come into contact with a CRISPR/nCas complex.

### CRISPR/nCas Composition for Cell Death—Comprising One or More Units of CRISPR/nCas System

The CRISPR/nCas composition for cell death comprises one or more units of a CRISPR/nCas system. A single unit of a CRISPR/nCas system may induce a single-strand break in a target nucleic acid comprising a specific nucleic acid sequence. The number of units of a CRISPR/nCas system comprised in the CRISPR/nCas composition for cell death varies depending on the number of target nucleic acids identified within the genome of a target cell and the number of different target nucleic acid sequences. Each of the CRISPR/nCas systems may independently be in the form of 1) a protein-RNA complex, 2) a vector or an encoding nucleic acid, 3) a composition, or 4) an appropriate combination of the foregoing forms. In other words, the CRISPR/nCas composition for cell death may comprise CRISPR/nCas systems in various forms.

For example, when four target nucleic acids are identified, and all of the target nucleic acids have different sequences, the CRISPR/nCas composition for cell death may comprise four units of a CRISPR/nCas system. In another example, when five target nucleic acids are identified, and all of the target nucleic acids have the same sequence, the CRISPR/nCas composition for cell death may comprise a single unit of a CRISPR/nCas system.

### Delivery Process of CRISPR/nCas Composition for Cell Death and PARP Inhibitor

### Overview of Delivery Process

The method for killing a target cell disclosed herein comprises a process of delivering a CRISPR/nCas composition for cell death and a PARP inhibitor to the target cell. Delivery of the CRISPR/nCas composition for cell death is performed to induce single-strand breaks at three or more target nucleic acids within the genome of the target cell. Delivery of the PARP inhibitor to the target cell is performed to inhibit PARP-mediated single-stranded nucleic acid repair within the target cell. As long as the above purposes can be achieved, the process of delivering the CRISPR/nCas composition for cell death and the process of delivering the PARP inhibitor can be implemented in various ways. For example, both the CRISPR/nCas composition for cell death and the PARP inhibitor may be delivered to the target cell together. In this case, the CRISPR/nCas composition for cell death and the PARP inhibitor may be co-formulated for use. In another example, the CRISPR/nCas composition for cell death and the PARP inhibitor may be delivered to the target cell independently of each other.

### Co-Formulated Delivery of CRISPR/nCas Composition for Cell Death and PARP Inhibitor

The processes of delivering the CRISPR/nCas composition for cell death and the PARP inhibitor may be implemented by delivering both to the target cell together. Furthermore, in order to deliver both the CRISPR/nCas composition for cell death and the PARP inhibitor together, the CRISPR/nCas composition for cell death and the PARP inhibitor may be co-formulated.

### Delivery of CRISPR/nCas Composition for Cell Death

The method for killing a target cell may be implemented as the processes of independently delivering the CRISPR/nCas composition for cell death and the PARP inhibitor to the target cell. Here, the method for delivering the CRISPR/nCas composition for cell death is not particularly limited as long as the purpose of delivering the CRISPR/nCas composition for cell death to the target cell can be achieved. The CRISPR/nCas composition for cell death can be delivered using known techniques as appropriate. For example, when the CRISPR/nCas composition for inducing cell death comprises a protein-RNA complex, it may be introduced into a target cell by a delivery method using electroporation, microinjection, cell-penetrating peptides, or lipid nanoparticles. In another example, when the CRISPR/nCas composition for inducing cell death comprisess a vector or a coding nucleic acid, it may be delivered to a target cell using a viral vector (e.g., adenovirus, adeno-associated virus (AAV), retrovirus, or lentivirus), or a non-viral delivery system (e.g., plasmids, liposomes, or polymer-based delivery carriers).

### Delivery of PARP Inhibitor

The method for killing a target cell may be implemented as the processes of independently delivering the CRISPR/nCas composition for cell death and the PARP inhibitor to the target cell. Here, the method for delivering the PARP inhibitor is not particularly limited as long as the purpose of delivering the PARP inhibitor to the target cell can be achieved. The PARP inhibitor can be delivered using known techniques as appropriate.

### Utilizing Cell Death Method Using CRISPR/nCas Composition for Cell Death and PARP Inhibitor

The cell death method disclosed herein can selectively induce only targeted cell death when three or more target nucleic acids specific to the target cell can be identified. Accordingly, this method for killing a target cell may be applied to the removal of tumor cells, senescent cells, or other cells in which mutations have occurred in the genome. For example, the cell death method may be applied to the treatment of cancer patients. In another example, the cell death method may be applied to the removal of senescent cells in a subject.

### Possible Embodiments of Present Disclosure

nCas Protein

### Embodiment 1. nCas Protein

A Cas protein having single-strand break activity, which is referred to as any one selected from the following:
a Cas protein having single-strand break activity; a Cas protein having nickase activity; a nickase; and an nCas protein.

### Embodiment 2. Examples of Cas Protein

The Cas protein of Embodiment 1, wherein the nCas protein is a Cas protein, a fragment thereof, or a variant thereof selected from the following:
Cas12a (Cpf1); Cas12b1 (C2c1); Cas12c (C2c3); Cas12e (CasX); Cas12d (CasY); Cas12g; Cas12h; Cas12i; Cas1; Cas1B; Cas2; Cas3; Cas4; Cas5; Cas6; Cas7; Cas8; Cas9 (also known as Csn1 and Csx12); Cas10; Csy1; Csy2; Csy3; Cse1; Cse2; Csc1; Csc2; Csa5; Csn2; Csm2; Csm3; Csm4; Csm5; Csm6; Cmr1; Cmr3; Cmr4; Cmr5; Cmr6; Csb1; Csb2; Csb3; Csx17; Csx14; Csx10; Csx16; CsaX; Csx3; Csx1; Csx15; Csf1; Csf2; Csf3; Csf4; Cas13a (C2c2); Cas13b; Cas13c; Cas13d; Cas14; xCas9; circularly permuted Cas9; and/or an Argonaute (Ago) domain.

### Embodiment 3. Limitation to Cas9

The Cas protein of Embodiment 2, wherein the nCas protein is a Cas9 protein, a fragment thereof, or a variant thereof.

### Embodiment 4. Limitation to SpCas9 Variant

The Cas protein of Embodiment 3, wherein the nCas protein is a variant of a Streptococcus pyogenes-derived Cas9 protein comprising a mutation selected from the following:
a D10A mutation with reference to the amino acid sequence of SEQ ID NO: 1; and an H840A mutation with reference to the amino acid sequence of SEQ ID NO: 1.

### Embodiment 5. Limitation to PAM

The Cas protein of Embodiment 4, wherein the nCas protein recognizes a 5′-NGG-3′ PAM.

### Embodiment 6. Limitation to Cas12a

The Cas protein of Embodiment 2, wherein the nCas protein is a Cas12a protein, a fragment thereof, or a variant thereof.

### Embodiment 7. Limitation to AsCas12a Variant

The Cas protein of Embodiment 6, wherein the nCas protein is a variant of an Acidaminococcus species-derived Cas12a protein comprising the following mutation:
an R1226A mutation with reference to the amino acid sequence of SEQ ID NO: 11.

### Embodiment 8. Limitation to nCas Protein Sequence

The Cas protein of any one of Embodiments 1 to 7, wherein the nCas protein comprises an amino acid sequence selected from:

```
  (SEQ ID NO: 2)
MDKKYSIGLAIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKM
KNYWRQLLNAKLITQRKFDNLTKAERGGLSELDKAGFIKRQLVETRQITKHVAQI
LDSRMNTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD,
  (SEQ ID NO: 3)
MDKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEE
DKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVD
QELDINRLSDYDVDAIVPQSFLKDDSIDNKVLTRSDKNRGKSDNVPSEEVVKKM
KNYWRQLLNAKLITQRKFDNLTKAERGGLSELDKAGFIKRQLVETRQITKHVAQI
LDSRMNTKYDENDKLIREVKVITLKSKLVSDFRKDFQFYKVREINNYHHAHDAYL
NAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHK
HYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD,
and
  (SEQ ID NO: 12)
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFI
GRTDNLTDAINKRHAEIYKGLFKAELFNGKVLKQLGTVTTTEHENALLRSFDKFT
TYFSGFYENRKNVFSAEDISTAIPHRIVQDNFPKFKENCHIFTRLITAVPSLREHF
ENVKKAIGIFVSTSIEEVFSFPFYNQLLTQTQIDLYNQLLGGISREAGTEKIKGLNE
VLNLAIQKNDETAHIIASLPHRFIPLFKQILSDRNTLSFILEEFKSDEEVIQSFCKYK
TLLRNENVLETAEALFNELNSIDLTHIFISHKKLETISSALCDHWDTLRNALYERRI
SELTGKITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARLTGIKL
EMEPSLSFYNKARNYATKKPYSVEKFKLNFQMPTLASGWDVNKEKNNGAILFV
KNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISF
QRIAEKEIMDAVETGKLYLFQIYNKDFAKGHHGKPNLHTLYWTGLFSPENLAKTS
IKLNGQAELFYRPKSRMKRMAHRLGEKMLNKKLKDQKTPIPDTLYQELYDYVNH
RLSHDLSDEARALLPNVITKEVSHEIIKDRRFTSDKFFFHVPITLNYQAANSPSKF
NQRVNAYLKEHPETPIIGIDRGERNLIYITVIDSTGKILEQRSLNTIQQFDYQKKLD
NREKERVAARQAWSVVGTIKDLKQGYLSQVIHEIVDLMIHYQAVVVLENLNFGF
KSKRTGIAEKAVYQQFEKMLIDKLNCLVLKDYPAEKVGGVLNPYQLTDQFTSFA
KMGTQSGFLFYVPAPYTSKIDPLTGFVDPFVWKTIKNHESRKHFLEGFDFLHYD
VKTGDFILHFKMNRNLSFQRGLPGFMPAWDIVFEKNETQFDAKGTPFIAGKRIV
PVIENHRFTGRYRDLYPANELIALLEEKGIVFRDGSNILPKLLENDDSHAIDTMVA
LIRSVLQMANSNAATGEDYINSPVRDLNGVCFDSRFQNPEWPMDADANGAYHI
ALKGQLLLNHLKESKDLKLQNGISNQDWLAYIQELRN.
```

### Embodiment 9. Inclusion of Nuclear Localization Signal (NLS)

The Cas protein of any one of Embodiments 1 to 8, wherein the nCas protein further comprises one or more Nuclear Localization Signals (NLS).

### Embodiment 10. Limitation to Structure Comprising NLS

The Cas protein of Embodiment 9, wherein the nCas protein has the following structure:

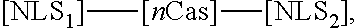

wherein NLS₁ is a first NLS or is absent,
nCas is the nCas protein of any one of Embodiments 1 to 8, and
NLS₂ is a second NLS or is absent,
wherein at least one of NLS₁ and NLS₂ must be present.

### Embodiment 11. Examples of NLS Sequence

The Cas protein of Embodiment 9 or 10,
wherein the NLS is an amino acid sequence selected from PKKKRKV (SEQ ID NO: 13), KRPAATKKAGQAKKKK (SEQ ID NO: 14), PAAKRVKLD (SEQ ID NO: 15), RQRRNELKRSP (SEQ ID NO: 16), NQSSNFGPMKGGNFGGRSSGPYGGGGQYFAKPRNQGGY (SEQ ID NO: 17), RMRIZFKNKGKDTAELRRRRVEVSVELRKAKKDEQILKRRNV (SEQ ID NO: 18), VSRKRPRP (SEQ ID NO: 19), PPKKARED (SEQ ID NO: 20), PQPKKKPL (SEQ ID NO: 21), SALIKKKKKMAP (SEQ ID NO: 22), DRLRR (SEQ ID NO: 23), PKQKKRK (SEQ ID NO: 24), RKLKKKIKKL (SEQ ID NO: 25), REKKKFLKRR (SEQ ID NO: 26), KRKGDEVDGVDEVAKKKSKK (SEQ ID NO: 27), and RKCLQAGMNLEARKTKK (SEQ ID NO: 28), or any combination of the foregoing amino acid sequences.

### Embodiment 12. Limitation to Similar Sequences

An nCas9 protein having an amino acid sequence that is identical to, matched with, or homologous to the nCas protein of any one of Embodiments 1 to 11 by about 80% or more, about 81% or more, about 82% or more, about 83% or more, about 84% or more, about 85% or more, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more, or about 99% or more.

### Guide RNA

### Embodiment 13. Guide RNA #1

A guide RNA comprising the following:
a scaffold; and
a guide domain,
wherein the guide domain targets a target nucleic acid.

### Embodiment 14. Guide RNA #2

A guide RNA comprising the following:
a tracrRNA; and
a crRNA comprising a direct repeat region and a guide domain,
wherein the tracrRNA and the direct repeat region of the crRNA are collectively referred to as a scaffold, and
the guide domain targets a target nucleic acid.

### Embodiment 15. Guide RNA #3

A guide RNA comprising the following:
a crRNA comprising a direct repeat region and a guide domain,
wherein the direct repeat region is referred to as a scaffold, and
the guide domain targets a target nucleic acid.

### Embodiment 16. Limitation to Scaffold Function

The guide RNA of any one of Embodiments 13 to 15, wherein the scaffold interacts with the nCas protein of any one of Embodiments 1 to 12, and
the guide RNA forms a complex with the nCas protein.

### Embodiment 17. Single Guide RNA or Dual Guide RNA

The guide RNA of any one of Embodiments 13 to 16, wherein the guide RNA is one RNA molecule or two RNA molecules.

### Embodiment 18. Targeting of Target Sequence

The guide RNA of any one of Embodiments 13 to 17,
wherein, when the target nucleic acid is a double-stranded DNA comprising a target strand and a non-target strand,
the guide domain targeting the target nucleic acid is selected from the following:
a guide domain having a sequence identical to, matched with, the same as, or equivalent to all or a part of a sequence of the non-target strand;
a guide domain being capable of hybridizing with or binding to the target strand; and
a guide domain having a sequence complementary to all or a part of a sequence of the target strand.

### Embodiment 19. Guide Domain Length

The guide RNA of any one of Embodiments 13 to 18,
wherein the guide domain has a length of
12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt, or
a length within a numerical range defined by the aforementioned two values (for example, in the range of 18 nt to 22 nt).

### Embodiment 20. Guide Domain Mismatch

The guide RNA of any one of Embodiments 13 to 19, wherein the guide domain is complementary to all or part of a sequence of the target nucleic acid,
but comprises 0, 1, 2, 3, 4, or 5 mismatches.

### Embodiment 21. Guide Domain Homology

The guide RNA of any one of Embodiments 13 to 20, wherein the guide domain is capable of hybridizing with or binding to the target nucleic acid, and
the guide domain has a sequence
that is identical to, matched with, the same as, or equivalent to the sequence of the target nucleic acid by about 80% or more, about 81% or more, about 82% or more, about 83% or more, about 84% or more, about 85% or more, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more, or about 99% or more.

### Embodiment 22. SpCas9 Guide RNA #1-Single Guide RNA

The guide RNA of any one of Embodiments 13 to 21, wherein the scaffold comprises a nucleic acid sequence selected from the following:
any one nucleic acid sequence selected from GUUUCAGAGCUAUGCUGGAAACAGCAUAGCAAGUUGAAAUAAGGCUAGUC CGUUAUCAACUUGAAAAAGUGGCACCGAGUCGGUGC (SEQ ID NO: 4), GUUUAAGAGCUAUGCUGGAAACAGCAUAGCAAGUUUAAAUAAGGCUAGUC CGUUAUCAACUUGAAAAAGUGGCACCGAGUCGGUGC (SEQ ID NO: 5), GUUUUAGAGCUAGAAAUAGCAAGUUAAAAUAAGGCUAGUCCGUUAUCAAC UUGAAAAAGUGGCACCGAGUCGGUGC (SEQ ID NO: 6) GCACCGACUCGGUGCCACUUUUUCAAGUUGAUAACGGACUAGCCUUAUUU UAACUUGCUAUUUCUAGCUCUAAAAC (SEQ ID NO: 7), and GUUUUAGAGCUAGAAAUAGCAAGUUAAAAUAAGGCUAGUCCGUUAUCAAC UUGAAAAAGUGGCACCGAGUCGGUGCUUUUUU (SEQ ID NO: 8); and
a sequence that is identical to, matched with, equivalent to, or similar to any one of the nucleic acid sequences selected from SEQ ID NOs: 4 to 8 by 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more.

### Embodiment 23. SpCas9 Guide RNA #2-Separate Definition of tracrRNA and Direct Repeat Region

The guide RNA of any one of Embodiments 13 to 22, wherein the scaffold comprises a tracrRNA and a direct repeat region,
wherein the tracrRNA comprises a nucleic acid sequence of UAGCAAGUUAAAAAAGGCUAGUCCGUUAUCAACUUGAAAAAGUGGCACCG AGUCGGUGC (SEQ ID NO: 9), or
comprises a sequence that is identical to, matched with, equivalent to, or similar to the nucleic acid sequence of SEQ ID NO: 9 by 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more, and
the direct repeat region comprises a nucleic acid sequence of GUUUUAGAGCUA (SEQ ID NO: 10), or
comprises a sequence that is identical to, matched with, equivalent to, or similar to the nucleic acid sequence of SEQ ID NO: 10 by 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more.

### CRISPR/nCas System

### Embodiment 24. CRISPR/nCas Complex

A CRISPR/nCas complex comprising the following:
the nCas protein of any one of Embodiments 1 to 12; and
the guide RNA of any one of Embodiments 13 to 23,
wherein a scaffold of the guide RNA interacts with the nCas protein to form a complex,
the CRISPR/nCas complex targets a target nucleic acid to induce a single-strand break, and
the CRISPR/nCas complex is also referred to as an RNA-protein complex, a RibonucleoProtein (RNP), or an RNA-guided nickase.

### Embodiment 25. CRISPR/nCas Vector

A vector expressing each component of a CRISPR/nCas system, the vector comprising the following:
a nucleic acid encoding the nCas protein of any one of Embodiments 1 to 12; and
a nucleic acid encoding the guide RNA of any one of Embodiments 13 to 23,
wherein the vector expressing each component of the CRISPR/nCas system is configured to express the nCas protein and the guide RNA in a cell,
the nCas Protein and the guide RNA are capable of forming a complex,
the CRISPR/nCas complex is capable of targeting a target nucleic acid to induce a single-strand break, and
the vector expressing each component of the CRISPR/nCas system is also referred to as a CRISPR/nCas-encoding nucleic acid or a CRISPR/nCas vector.

### Embodiment 26. Viral Vector

The vector of Embodiment 25, wherein the CRISPR/nCas vector is a viral vector.

### Embodiment 27. Examples of Viral Vector

The vector of Embodiment 26, wherein the viral vector is selected from the following:
lentivirus; adeno-associated virus (AAV); adenovirus; retrovirus; vaccinia virus; poxvirus; herpes simplex virus (HSV); or any combination thereof.

### Embodiment 28. Non-Viral Vector

The vector of Embodiment 25, wherein the CRISPR/nCas vector is a non-viral vector.

### Embodiment 29. Examples of Non-Viral Vector

The vector of Embodiment 28, wherein the non-viral vector is selected from the following:
plasmid; phage; naked DNA; DNA complex; circular single-stranded DNA; mRNA; or any combination thereof.

### Embodiment 30. CRISPR/nCas Composition

A CRISPR/nCas composition comprising the following:
the nCas protein of any one of Embodiments 1 to 12, or a nucleic acid encoding the same; and
the guide RNA of any one of Embodiments 13 to 23, or a nucleic acid encoding the same,
wherein the nCas Protein and the guide RNA are capable of forming a complex, and
the CRISPR/nCas complex is capable of targeting a target nucleic acid to induce a single-stranded nucleic acid break.

### Embodiment 31. Inclusion of nCas Protein mRNA and Guide RNA

The CRISPR/nCas composition of Embodiment 30, wherein the CRISPR/nCas composition comprises a RNA encoding the Cas protein, and the guide RNA.

### Embodiment 32. Single Unit of CRISPR/nCas System

A CRISPR/nCas system comprising the following:
the CRISPR/nCas complex of Embodiment 24; the CRISPR/nCas vector of any one of Embodiments 25 to 29; or the CRISPR/nCas composition of Embodiment 30 or 31,
wherein the CRISPR/nCas system comprises one type of nCas protein or a nucleic acid encoding the same, and one type of guide RNA or a nucleic acid encoding the same.

### CRISPR/nCas Composition for Cell Death

### Embodiment 33. CRISPR/nCas Composition for Cell Death

A CRISPR/nCas composition for cell death, the CRISPR/nCas composition comprising the following:
one or more units (types) of the CRISPR/nCas system of Embodiment 32,
wherein a single unit of the CRISPR/nCas system refers to a CRISPR/nCas system comprising one type of nCas protein or a nucleic acid encoding the same, and one type of guide RNA or a nucleic acid encoding the same.

### PARP Inhibitor

### Embodiment 34. PARP Inhibitor

A PARP inhibitor.

### Embodiment 35. Key Function of PARP Inhibitor

The PARP inhibitor of Embodiment 34, wherein the PARP inhibitor inhibits PARP-mediated DNA repair mechanisms.

### Embodiment 36. Examples of PARP Inhibitor

The PARP inhibitor of Embodiment 34 or 35, wherein the PARP inhibitor is selected from the following:
olaparib; rucaparib; niraparib; veliparib; iniparib; pamiparib; fluzoparib; talazoparib; CEP-9722; E7449 (2X-121); BGB-290; AZD2461; 3-aminobenzamide; INO-1001; or any combination thereof.

### Embodiment 37. Pharmaceutical Composition for Oral Administration Comprising PARP Inhibitor

A pharmaceutical composition comprising the following:
the PARP inhibitor of any one of Embodiments 34 to 36; and
a pharmaceutically acceptable carrier suitable for oral administration.

### Embodiment 38. Limitation to Carrier for Oral Administration

The pharmaceutical composition of Embodiment 37, wherein the pharmaceutically acceptable carrier suitable for oral administration is selected from the following:
diluents/fillers, for example, microcrystalline cellulose, anhydrous lactose, lactose monohydrate, mannitol, corn starch, potato starch, pregelatinized starch, dicalcium phosphate, and/or sorbitol; binders, for example, povidone (PVP), hydroxypropyl methylcellulose (HPMC), methylcellulose, ethyl cellulose, gelatin, and/or gum acacia; disintegrants, for example, sodium starch glycolate, croscarmellose sodium, crospovidone, and/or low-substituted hydroxypropyl cellulose (L-HPC); lubricants, for example, magnesium stearate, stearic acid, sodium stearyl fumarate, and/or talc; glidants, for example, colloidal silicon dioxide, and/or talc; coating materials, for example, HPMC, methacrylic acid copolymers (EUDRAGIT®), ethyl cellulose, and/or polyvinyl alcohol (PVA); plasticizers, for example, polyethylene glycol (PEG), triacetin, and/or propylene glycol; sweeteners, for example, sucrose, aspartame, sucralose, and/or xylitol; flavoring agents, for example, natural and artificial flavorants; colorants, for example, iron oxide pigments, FD&C dyes, and/or titanium dioxide; pH adjusters, for example, citric acid, sodium citrate, and/or tartaric acid; preservatives, for example, sodium benzoate, and/or potassium sorbate; solubilizers, for example, PEG, and/or polysorbates (Tween®); absorption enhancers, for example, sodium lauryl sulfate, and/or lecithin; sustained-release matrices, for example, HPMC, ethyl cellulose, and/or carbomers; enteric coating polymers, for example, cellulose acetate phthalate, and/or hydroxypropyl methylcellulose phthalate; mucoadhesive polymers, for example, carbopol, and/or chitosan; and antioxidants, for example, butylated hydroxyanisole (BHA), butylated hydroxytoluene (BHT), and/or ascorbic acid.

### Embodiment 39. Pharmaceutical Composition for Injection Comprising PARP Inhibitor

A pharmaceutical composition comprising the following:
the PARP inhibitor of any one of Embodiments 34 to 36; and
a pharmaceutically acceptable carrier suitable for injection.

### Embodiment 40. Limitation to Carrier for Injection

The pharmaceutical composition of Embodiment 39, wherein the pharmaceutically acceptable carrier suitable for injection is selected from the following:
aqueous vehicles, for example, water for injection, normal saline, Ringer's solution, and/or dextrose solution; non-aqueous vehicles, for example, ethyl oleate, isopropyl myristate, vegetable oils (such as sesame oil, corn oil, and peanut oil), and/or medium-chain triglycerides; cosolvents, for example, propylene glycol, ethanol, PEG, and/or glycerin; surfactants/emulsifiers, for example, polysorbates (such as Tween 80 and Tween 20), sorbitan esters (such as Span), lecithin, and/or sodium deoxycholate; pH adjusters, for example, sodium hydroxide, hydrochloric acid, citric acid/sodium citrate buffer, and/or phosphate buffers; tonicity adjusters, for example, sodium chloride, dextrose, mannitol, and/or glycerin; preservatives, for example, benzyl alcohol, phenol, methylparaben, and/or propylparaben; antioxidants, for example, sodium metabisulfite, ascorbic acid, alpha-tocopherol, and/or BHA; chelating agents, for example, ethylenediaminetetraacetic acid (EDTA), and/or citric acid; bulking agents (for lyophilized formulations), for example, mannitol, lactose, trehalose, and/or sucrose; polymers for sustained release, for example, poly(lactic-co-glycolic acid) (PLGA), polylactic acid (PLA), and/or chitosan; viscosity enhancers, for example, sodium carboxymethylcellulose, HPMC, and/or polyvinylpyrrolidone (PVP); liposomal components, for example, phospholipids (such as phosphatidylcholine), and/or cholesterol; nanoparticle components, for example, albumin, and/or poloxamers; cryoprotectants, for example, trehalose, sucrose, and/or glycine; local anesthetics (for reducing injection pain), for example, lidocaine, and/or benzyl alcohol; solubilizers, for example, cyclodextrins (such as β-cyclodextrin and hydroxypropyl-β-cyclodextrin), and/or Cremophor® EL; gases for suspension (in the case of microspheres), for example, nitrogen, and/or carbon dioxide; or any combination thereof.

### Embodiment 41. PARP Inhibitor Concentration

The PARP inhibitor of any one of Embodiments 34 to 40,
wherein the PARP inhibitor has a concentration at which cytotoxicity is not observed when administered alone.

### Composition for Inducing Targeted Cell Death Comprising CRISPR/nCas Composition for Cell Death and PARP Inhibitor

### Embodiment 42. Composition for Inducing Targeted Cell Death

A composition for inducing targeted cell death, the composition comprising the following:
the CRISPR/nCas composition for inducing targeted cell death of Embodiment 33; and
a PARP inhibitor of any one of Embodiments 34 to 36 and 41.

### Embodiment 43. Addition of Carrier

The composition of Embodiment 42, wherein the composition for inducing targeted cell death further comprises a pharmaceutically acceptable carrier.

### Embodiment 44. Examples of Carrier

The composition of Embodiment 43, wherein the pharmaceutically acceptable carrier is selected from the following:
aqueous vehicles, for example, water for injection, normal saline, Ringer's solution, and/or dextrose solution; non-aqueous vehicles, for example, ethyl oleate, isopropyl myristate, vegetable oils (such as sesame oil, corn oil, and peanut oil), and/or medium-chain triglycerides; cosolvents, for example, propylene glycol, ethanol, PEG, and/or glycerin; surfactants/emulsifiers, for example, polysorbates (such as Tween 80 and Tween 20), sorbitan esters (such as Span), lecithin, and/or sodium deoxycholate; pH adjusters, for example, sodium hydroxide, hydrochloric acid, citric acid/sodium citrate buffer, and/or phosphate buffers; tonicity adjusters, for example, sodium chloride, dextrose, mannitol, and/or glycerin; preservatives, for example, benzyl alcohol, phenol, methylparaben, and/or propylparaben; antioxidants, for example, sodium metabisulfite, ascorbic acid, alpha-tocopherol, and/or BHA; chelating agents, for example, EDTA, and/or citric acid; bulking agents (for lyophilized formulations), for example, mannitol, lactose, trehalose, and/or sucrose; polymers for sustained release, for example, PLGA, PLA, and/or chitosan; viscosity enhancers, for example, sodium carboxymethylcellulose, HPMC, and/or PVP; liposomal components, for example, phospholipids (such as phosphatidylcholine), and/or cholesterol; nanoparticle components, for example, albumin, and/or poloxamers; cryoprotectants, for example, trehalose, sucrose, and/or glycine; local anesthetics (for reducing injection pain), for example, lidocaine, and/or benzyl alcohol; solubilizers, for example, cyclodextrins (such as β-cyclodextrin and hydroxypropyl-β-cyclodextrin), and/or Cremophor® EL; gases for suspension (in the case of microspheres), for example, nitrogen, and/or carbon dioxide; or any combination thereof.

### Method for Killing Target Cell Using CRISPR/nCas Composition for Cell Death and PARP Inhibitor

### Embodiment 45. Method for Killing Target Cell

A method for killing a target cell, the method comprising the following:
a process of delivering the CRISPR/nCas composition of Embodiment 33 and the PARP inhibitor of any one of Embodiments 34 to 36 and 41, to the target cell,
wherein the CRISPR/nCas composition targets three or more target nucleic acids within the genome of the target cell and induces single-strand breaks, and
the CRISPR/nCas Composition and the PARP inhibitor act together to induce targeted cell death.

### Embodiment 46. Method for Killing Target Cell #2-N Target Nucleic Acids

The method of Embodiment 45, wherein the target cell comprises N target nucleic acids within its genome,
the N target nucleic acids are distinguished into a total of m nucleic acid sequences,
N is an integer of 3 or greater, and m is an integer of 1 or greater and N or less, and
the CRISPR/nCas composition for cell death comprises m units of the CRISPR/nCas system of Embodiment 32.

### Embodiment 47. Three or More Target Nucleic Acids

The method of Embodiment 45 or 46, wherein the target cell in a patient comprises a first target nucleic acid, a second target nucleic acid, and a third target nucleic acid within its genome.

### Embodiment 48. Inclusion of Single Unit of CRISPR/nCas System

The method of Embodiment 47,
wherein the CRISPR/nCas composition for cell death comprises a first CRISPR/nCas system, and,
when the first, second, and third target nucleic acids have the same nucleic acid sequence, and such a nucleic acid sequence is hereinafter referred to as a first target sequence,
a guide domain of a guide RNA of the first CRISPR/nCas system targets the first target sequence, and
the first CRISPR/nCas system recognizes the first target nucleic acid and induces a single-strand break.

### Embodiment 49. Inclusion of Two Units of CRISPR/nCas System

The method of Embodiment 47,
wherein the CRISPR/nCas for cell death comprises a first CRISPR/nCas system and a second CRISPR/nCas system, and,
when the first and second target nucleic acids have the same nucleic acid sequence, such a nucleic acid sequence is hereinafter referred to as a first target sequence, and
the third target nucleic acid has a nucleic acid sequence that is hereinafter referred to as a second target sequence,
a guide domain of a guide RNA of the first CRISPR/nCas system targets the first target sequence,
the first CRISPR/nCas system recognizes the first target nucleic acid and induces a single-strand break,
a guide domain of a guide RNA of the second CRISPR/nCas system targets the second target sequence, and
the second CRISPR/nCas system recognizes the second target nucleic acid and induces a single-strand break.

### Embodiment 50. Inclusion of Three Units of CRISPR/nCas System

The method of Embodiment 47,
wherein the CRISPR/nCas for cell death comprises a first CRISPR/nCas system, a second CRISPR/nCas system, and a third CRISPR/nCas system, and,
when the first target nucleic acid has a nucleic acid sequence that is hereinafter referred to as a first target sequence,
the second target nucleic acid has a nucleic acid sequence that is hereinafter referred to as a second target sequence, and
the third target nucleic acid has a nucleic acid sequence that is hereinafter referred to as a third target sequence,
a guide domain of a guide RNA of the first CRISPR/nCas system targets the first target sequence,
the first CRISPR/nCas system recognizes the first target nucleic acid and induces a single-strand break,
a guide domain of a guide RNA of the second CRISPR/nCas system targets the second target sequence,
the second CRISPR/nCas system recognizes the second target nucleic acid and induces a single-strand break,
a guide domain of a guide RNA of the third CRISPR/nCas system targets the third target sequence, and
the third CRISPR/nCas system recognizes the third target nucleic acid and induces a single-strand break.

### Embodiment 51. Alternative Expression for Delivery [000439]

The method of any one of Embodiments 45 to 50,
wherein, in the process of delivering the CRISPR/nCas composition for cell death and the PARP inhibitor (hereinafter referred to as the above components) to the target cell,
the expression “delivering the above components to the target cell” is replaced with the following expression:
injecting the above components into the target cell; introducing the above components into the target cell; administering the above components to the target cell; importing the above components into the target cell; transporting the above components into the target cell; causing the target cell to absorb the above components; causing the above components to penetrate the target cell; inducing entry of the above components into the target cell; infusing the above components into the target cell; or any combination thereof.

### Embodiment 52. Combined Delivery of the CRISPR/nCas Composition for Cell Death and the PARP Inhibitor

The method of any one of Embodiments 45 to 51, wherein the process of delivering the CRISPR/nCas composition and the PARP inhibitor to the target cell
is performed by delivering the composition for inducing targeted cell death of any one of Embodiments 42 to 44 to the target cell.

### Embodiment 53. Independent Delivery of CRISPR/nCas Composition for Cell Death and PARP Inhibitor

The method of any one of Embodiments 45 to 51, wherein the process of delivering the CRISPR/nCas composition and the PARP inhibitor to the target cell
is performed by independently delivering the CRISPR/nCas composition and the PARP inhibitor to the target cell.

### Embodiment 54. Delivery of CRISPR/nCas Composition for Cell Death

The method of Embodiment 53, wherein the CRISPR/nCas composition is delivered to the target by a method selected from the following:
lipid nanoparticle-mediated transfection; electroporation; gene gun; sonoporation; magnetofection; microinjection; cell squeezing; lipofection; cationic liposome-mediated transfection; lithium acetate-DMSO; DEAE-dextran-mediated transfection; polyethyleneimine (PEI)-mediated transfection; nanoparticle-mediated nucleic acid delivery; or any combination of the foregoing delivery methods.

### Method for Selectively Killing Target Cell in Patient

### Embodiment 55. Method for Selectively Killing Target Cell

A method for selectively killing a target cell in a patient,
wherein the target cell in the patient has three or more target nucleic acids within its genome, and non-target cells in the patient do not have the target nucleic acid,
wherein the method comprises the following:
a process of administering the CRISPR/nCas composition for cell death of Embodiment 33 and the PARP inhibitor of any one of Embodiments 34 to 36 and 41 to the patient,
wherein the CRISPR/nCas composition for cell death targets three or more target nucleic acids within the genome of the target cell in the patient and induces single-strand breaks, and
the CRISPR/nCas composition for cell death and the PARP inhibitor act together to selectively induce targeted cell death.

### Embodiment 56. N Target Nucleic Acids and m Different Targets

The method of Embodiment 55, wherein the target cell in the patient has N target nucleic acids, and
the N target nucleic acids are distinguished into a total of m nucleic acid sequences,
wherein N is an integer of 3 or greater, and m is an integer of 1 or greater and N or less, and
the CRISPR/nCas composition for cell death comprises m units of the CRISPR/nCas system of Embodiment 32.

### Embodiment 57. Three or More Target Nucleic Acids

The method of Embodiment 55 or 56, wherein the target cell in the patient comprises a first target nucleic acid, a second target nucleic acid, and a third target nucleic acid within its genome.

### Embodiment 58. Inclusion of Single Unit of CRISPR/nCas System

The method of Embodiment 57,
wherein the CRISPR/nCas composition for cell death comprises a first CRISPR/nCas system, and,
when the first, second, and third target nucleic acids have the same nucleic acid sequence, and such a nucleic acid sequence is hereinafter referred to as a first target sequence,
a guide domain of a guide RNA of the first CRISPR/nCas system targets the first target sequence, and
the first CRISPR/nCas system recognizes the first target nucleic acid and induces a single-strand break.

### Embodiment 59. Inclusion of Two Units of CRISPR/nCas System

The method of Embodiment 57,
wherein the CRISPR/nCas for cell death comprises a first CRISPR/nCas system and a second CRISPR/nCas system, and,
when the first and second target nucleic acids have the same nucleic acid sequence, such a nucleic acid sequence is hereinafter referred to as a first target sequence, and
the third target nucleic acid has a nucleic acid sequence that is hereinafter referred to as a second target sequence,
a guide domain of a guide RNA of the first CRISPR/nCas system targets the first target sequence, and
the first CRISPR/nCas system recognizes the first target nucleic acid and induces a single-strand break,
a guide domain of a guide RNA of the second CRISPR/nCas system targets the second target sequence, and
the second CRISPR/nCas system recognizes the second target nucleic acid and induces a single-strand break.

### Embodiment 60. Inclusion of Three Units of CRISPR/nCas System

The method of Embodiment 57,
wherein the CRISPR/nCas for cell death comprises a first CRISPR/nCas system, a second CRISPR/nCas system, and a third CRISPR/nCas system, and,
when the first target nucleic acid has a nucleic acid sequence that is hereinafter referred to as a first target sequence,
the second target nucleic acid has a nucleic acid sequence that is hereinafter referred to as a second target sequence, and
the third target nucleic acid has a nucleic acid sequence that is hereinafter referred to as a third target sequence,
a guide domain of a guide RNA of the first CRISPR/nCas system targets the first target sequence, and
the first CRISPR/nCas system recognizes the first target nucleic acid and induces a single-strand break,
a guide domain of a guide RNA of the second CRISPR/nCas system targets the second target sequence,
the second CRISPR/nCas system recognizes the second target nucleic acid and induces a single-strand break,
a guide domain of a guide RNA of the third CRISPR/nCas system targets the third target sequence, and
the third CRISPR/nCas system recognizes the third target nucleic acid and induces a single-strand break.

### Embodiment 61. Alternative Expression for Administration

The method of any one of Embodiments 55 to 60,
wherein, in the process of administering the CRISPR/nCas composition for cell death and the PARP inhibitor (hereinafter referred to as the above components) to the patient,
the expression “administering the above components to the patient” is replaced with the following expression:
giving the above components to the patient; providing the above components to the patient; injecting the above components into the patient; medicating the patient with the above components; applying the above components to the patient; treating the patient with the above components; using the above components for the patient; dispensing the above components to the patient; supplying the above components to the patient; or any combination thereof.

### Embodiment 62. Limitation to Target Cell

The method of any one of Embodiments 55 to 61,
wherein the target cell in the patient is selected from the following:
cancer cell; benign tumor; malignant tumor; senescent cell; virus-infected cell; cell associated with immune diseases; cell having genomic sequence variations; or any combination thereof.

### Embodiment 63. Administration Method to Patient-Co-Administration

The method of any one of Embodiments 55 to 62, wherein the process of administering the CRISPR/nCas composition and the PARP inhibitor
is performed by administering the composition for inducing targeted cell death of any one of Embodiments 42 to 44 to the patient.

### Embodiment 64. Administration Method to Patient-Independent Administration

The method of any one of Embodiments 55 to 62, wherein the process of administering the CRISPR/nCas composition and the PARP inhibitor
is performed by independently administering the CRISPR/nCas composition and the PARP inhibitor to the patient.

### Embodiment 65. Administration Method of CRISPR/nCas Composition to Patient

The method of Embodiment 64, wherein the administration of the CRISPR/nCas composition to the patient is performed by a method selected from the following:
administration of a lipid nanoparticle comprising the CRISPR/nCas complex of Embodiment 24 to the patient; administration of a lipid nanoparticle comprising the CRISPR/nCas composition of Embodiment 31 to the patient; administration of the CRISPR/nCas vector of any one of Embodiments 25 to 29 to the patient; or any combination of the foregoing administration methods.

### Embodiment 66. Administration Method of PARP Inhibitor to Patient

The method of Embodiment 64 or 65, wherein the administration of the PARP inhibitor to the patient is performed by a method selected from the following:
oral administration of the pharmaceutical composition of Embodiment 37 or 38 to the patient; injection of the pharmaceutical composition of Embodiment 39 or 40 into the patient; or any combination of the foregoing administration methods.

### Method for Treating Cancer Using CRISPR/nCas Composition for Cell Death and PARP Inhibitor

### Embodiment 67. Method for Treating Cancer

A method for treating a cancer patient, the method comprising: performing the method of any one of Embodiments 55 to 66,
wherein the target cell of the patient is a cancer cell, and the target nucleic acids have nucleic acid sequences specific to the cancer cell.

### Embodiment 68. Limitation to Cancer Cell

The method of Embodiment 67, wherein the cancer cell is selected from the following:
melanoma; small cell lung cancer; non-small cell lung cancer;
glioma; liver cancer; thyroid tumor; stomach cancer; prostate cancer; breast cancer; ovarian cancer; bladder cancer; lung cancer; colorectal cancer; breast cancer; prostate cancer; glioblastoma; endometrial cancer; kidney cancer; colon cancer; pancreatic cancer; esophageal carcinoma; head and neck cancer; mesothelioma; sarcoma; osteosarcoma; cholangiocarcinoma; epidermal cancer; or any combination thereof.

### Embodiment 69. Nucleic Acid Sequence Specific to Cancer Cell

The method of Embodiment 67, wherein the cancer cell comprises a first target nucleic acid, a second target nucleic acid, and a third target nucleic acid within its genome, and
the first, second, and third target nucleic acids are nucleic acid sequences specific to the cancer cell and are not comprised in the genome of normal cells.

### Embodiment 70. Different Target Nucleic Acid Sequences

The method of Embodiment 68 or 69, wherein the first, second, and third target nucleic acids have different nucleic acid sequences.

### Embodiment 71. Same Target Nucleic Acid Sequence

The method of Embodiment 68 or 69, wherein the first, second, and third target nucleic acids have the same nucleic acid sequence.

### Pharmaceutical Composition

### Embodiment 72. Pharmaceutical Composition for Co-Administration with Anticancer Agent

A pharmaceutical composition for co-administration with an anticancer agent, the pharmaceutical composition comprising the following:
the CRISPR/nCas composition for inducing targeted cell death of Embodiment 33,
wherein the CRISPR/nCas composition for inducing targeted cell death targets three or more target nucleic acids within the genome of a target cancer cell and induces single-strand breaks; and
a pharmaceutically acceptable carrier,
wherein the anticancer agent comprises, as an active ingredient, the PARP inhibitor of any one of Embodiments 34 to 36 and 41.

### Embodiment 73. Pharmaceutical Composition for Cancer Treatment

A pharmaceutical composition for cancer treatment, the pharmaceutical composition comprising the composition of any one of Embodiments 42 to 44,
wherein a CRISPR/nCas composition for inducing targeted cell death of this composition targets three or more target nucleic acids within the genome of a target cancer cell and induces single-strand breaks.

### Use of CRISPR/nCas Composition for Cell Death and PARP Inhibitor

### Embodiment 74. Use of CRISPR/nCas Composition for Cell Death

A use of the CRISPR/nCas Composition for Cell Death of Embodiment 33 in the method of any one of Embodiments 45 to 66.

### Embodiment 75. Use of CRISPR/nCas Composition for Cell Death in Manufacture of Pharmaceutical

A use of the CRISPR/nCas composition for cell death of Embodiment 33 for a manufacture of a medication for co-administration with an anticancer agent,
wherein the anticancer agent comprises, as an active ingredient, the PARP inhibitor of any one of Embodiments 34 to 36 and 41.

### Embodiment 76. Use of PARP Inhibitor

A use of the PARP inhibitor of any one of Embodiments 34 to 36 and 41, the pharmaceutical composition of Embodiment 37 or 38, or the pharmaceutical composition of Embodiment 39 or 40 in the method of any one of Embodiments 45 to 66.

### EXPERIMENTAL EXAMPLE

Hereinafter, the present disclosure provided hereby will be described in more detail through Examples and Experimental Examples. The following examples are only for illustrating the content disclosed herein, and it is apparent to those skilled in the art that the scope of the present disclosure should not be construed as limited by these examples.

### Experimental Example 1. Experimental Methods and Materials

### Experimental Example 1.1. Cell Culture

HeLa (ATCC, CCL-2), HCT-116 (ATCC, CCL-247), and MDA-MB-231 (ATCC, HTB-26) cells were cultured in Dulbecco's Modified Eagle Medium (DMEM) (Gibco, #11965092). SW-480 cells (ATCC, CCL-228) were cultured in Roswell Park Memorial Institute (RPMI) 1640 medium (Gibco, #11875093). All cell culture media were supplemented with 10% fetal bovine serum (Gibco, #10082147) and 1% penicillin-streptomycin (Gibco, #15140122). Cell culture was performed at 37° C. in a humidified atmosphere containing 5% CO2. For the delivery of CRISPR RNP or RNA, 0.5×10⁴ cells were seeded onto 24-well plates one day before transfection.

### Experimental Example 1.2. Expression and Purification of Cas9-WT and nCas9-D10A

The pET-LbCas12a-ultra vector was constructed using a DNA sequence encoding LbCas12a-ultra23 and cloned into the pET28a+ vector (Addgene, #69864-3). The pET-Cas9-HN was derived from p3s-Cas9-HN (Addgene, #104171). The above vectors were transformed into the C3013 strain (NEB, #C30131), and recombinant Cas9WT or LbCas12a-ultra proteins were expressed by induction with 0.5 mM IPTG at 25° C. for 4 hours. Then, Ni-NTA agarose beads (Qiagen, #30210) were used for purification. The purified recombinant proteins were dialyzed against a dialysis buffer (20 mM HEPES, 150 mM KCl, 1 mM DTT, and 10% glycerol) and then concentrated using Amicon Ultra-4 50K filter units (Millipore, #UFC8050). The concentrated proteins were analyzed by SDS-PAGE and quantified by comparison with band intensities of a BSA standard.

### Experimental Example 1.3. Preparation of sgRNA

sgRNAs for SpCas9WT and LbCas12a-ultra were transcribed in vitro from DNA templates comprising a T7 promoter and an sgRNA sequence using the HiScribe T7 Quick High Yield RNA Synthesis Kit (NEB, #E2050S. The DNA templates were generated by extension PCR using 100 μM of each primer and NEBNext High-Fidelity 2×PCR Master Mix (NEB, #M0541L). Using the DNA templates, sgRNAs were synthesized according to the manufacturer's standard RNA synthesis protocol. The synthesized sgRNAs were purified using the Monarch RNA Cleanup Kit (NEB, #T2040L) and evaluated by visualization on a denaturing agarose gel or a PAGE gel.

### Experimental Example 1.4. Preparation of Cas9 mRNA

To synthesize Cas9WT mRNA, a DNA template was generated by digesting p3s-Cas9HC (Addgene, #43945) with restriction enzymes Spel-HF (NEB, #R3133S) and Xhol (NEB, #R0146S). Using the DNA template, mRNA was synthesized using the NEB HiScribe T7 ARCA mRNA Kit with tailing (NEB, #E2060S). The synthesized mRNA was purified using the Monarch RNA Cleanup Kit (NEB, #T2040L) and evaluated by visualization on a denaturing agarose gel or a PAGE gel. The Cas9WT mRNA was validated using an sgRNA targeting a single site.

### Experimental Example 1.5. Preparation of RNP and LNP Comprising Same

For the delivery of RNP, lipid nanoparticles (LNPs) were prepared using an ethanol dilution method. All lipids (the molar ratio of C12-200/DOPE/cholesterol/DMG-PEG/DOTAP in the LNPs was fixed at 35/16/46.5/2.5/11.11) were dissolved in ethanol. RNPs (with a molar ratio of sgRNA to Cas9 protein of 1:1) were dissolved in PBS (pH 7.4). The two solutions were rapidly mixed by pipetting at a PBS-to-ethanol ratio of 3:1 (v/v) and a total lipid-to-sgRNA ratio of 20:1 (w/w), followed by incubation at room temperature for 15 minutes. For in vivo experiments, the RNP-loaded LNP formulation was purified by dialysis against PBS for 2 hours using a dialysis kit (Pur-A-Lyzer Midi Dialysis Kits, MWCO 3.5 kDa) and then concentrated using Amicon Ultra-15 centrifugal filters (MWCO 50 kDa), followed by intratumoral injection (50 μg of sgRNA-loaded LNP per injection). The above C12-200 was purchased from Cayman Chemical. The above DOPE, DOTAP, and DMG-PEG (MW 2000) were purchased from Avanti Polar Lipids. The above Cholesterol, the Pur-A-Lyzer Midi Dialysis Kit (MWCO, 3.5 kDa), and the Amicon Ultra-15 centrifugal filters (MWCO, 50 kDa) were purchased from Sigma. All chemicals were used without further purification.

### Experimental Example 1.6. Treatment with PARP Inhibitor

A total of 0.5×10⁴ cells were mixed with 1 μM olaparib (AZD2281) (Selleckchem, #S1060), 1 μM rucaparib (Selleckchem, #S4948), 0.1 μM niraparib (MK-4827) (Selleckchem, #S2741), 2 μM veliparib (ABT-888) (Selleckchem, #S1004), or 10 UM iniparib (BSI-201) (Selleckchem, #S1087), and seeded onto 24-well plates. Under each of the PARP inhibitors at the above concentrations, no observable cytotoxicity was detected when the cells were measured using the CellTiter-Glo assay.

### Experimental Example 1.7. RNP Transfection

To transfect RNPs, cells were transfected with 665 ng of the Cas protein of each Embodiment and 95 ng of the sgRNA of each Embodiment using a Lipofectamine CRISPRMAX Cas9 Transfection Reagent (Invitrogen, #CMAX00015), and then cultured for 3 days. When treated together with a PARP inhibitor, the PARP inhibitor was treated according to Experimental Example 1.6, and the RNPs were treated one day thereafter.

### Experimental Example 1.8. Measurement of Cell Viability

Cell viability was measured using a CellTiter-Glo Luminescent Cell Viability Assay (Promega, #G7571). After the transfected cells were cultured for 3 days, the cells were detached using Trypsin-EDTA (0.25%) (Gibco, #25200056). The cells were diluted in 400 μL of DMEM, followed by dispensing 100 μL of the cells, mixed with 100 μL of the CellTiter-Glo Reagent, and then incubated at room temperature for 10 minutes. Luminescence signals were measured using an Infinite 200 (Tecan) in a white opaque 96-well plate (Corning, #3917).

### Experimental Example 1.9. Colony Formation Assay

After the transfected cells were cultured for 3 days, the cells were harvested using Trypsin-EDTA (0.25%) (Gibco, #25200056). The harvested cells were seeded onto 6-well plates at the following densities: for Hela cells, 100 cells per well, 1,000 cells per well, and 10,000 cells per well; and for HCT-116 cells, 30 cells per well, 300 cells per well, and 3,000 cells per well. The cell culture medium was replaced every 5 days until 2 weeks after transfection. Thereafter, the cells were fixed with 4% paraformaldehyde at room temperature for 20 minutes, washed twice with PBS, stained with 0.005% crystal violet for 30 minutes, and washed with distilled water before scanning.

### Experimental Example 1.10. Targeted Sequencing and Analysis

To measure the frequency of CRISPR-induced indels, the following procedures were performed.

Cells were transfected with RNPs according to Experimental Example 1.7. After 3 days, the cells were harvested, and genomic DNA was extracted using a DNeasy Blood & Tissue Kit (Qiagen, #69506). A DNA library for targeted sequencing was prepared using primers comprising Illumina adapter sequences. The DNA library was quantified and loaded onto an Illumina NextSeq 500 or NovaSeq X System with 150 bp paired-end reads. Mutation frequencies at the target sites were analyzed using CRISPResso2 (v2.2.12) or CRISPRpic, and no significant difference was observed between the two analysis methods. The mutation frequency was calculated based on the number of reads comprising mutated indels or mutated sequences. Sequences corresponding to heterozygous alleles were classified as unmutated sequences.

### Experimental Example 1.11. Establishment of Tumor Xenograft Model

Female BALB/c nude mice (5 weeks old) were purchased from Orient Bio and used for all animal experiments. The mice were handled in accordance with the guidelines of the Institutional Animal Care and Use Committee of Ulsan National Institute of Science and Technology (UNISTIACUC-23-09). In the case of the xenograft model, 5×10⁶ HCT116 cells stably expressing Cas9WT were suspended in 60 μL of PBS (pH 7.4) and mixed with 60 μL of Matrigel (BD Bioscience), followed by subcutaneous injection into the flank of each mouse. Tumor volume and body weight were measured every two days. Tumor volume was calculated using the following formula.

$$V = \frac{{( a \times b )}^{2}}{2}$$

In the above formula, V represents the volume, a represents the length of the long axis, and b represents the length of the short axis.

When the tumor volume reached approximately 80 mm³, the mice were randomly divided into multiple groups for experimentation.

### Experimental Example 1.12. Tumor Growth Inhibition Experiment

To evaluate the antitumor efficacy of the administered materials in each group, mice bearing tumors established according to Experimental Example 1.11 were anesthetized with 1.5% isoflurane, and the administered material (PBS or LNP) was intratumorally injected every two days. A total of eight injections were performed over a period of 14 days, and the mice were sacrificed on day 25.

The tumor growth inhibition rate was calculated using the following formula.

$$Tumor growth inhibitation rate ( \% ) = ( 1 - \frac{{V}_{Day 0 , treatment} - {V}_{Day 24 , treatment}}{{V}_{Day 0 , control} - {V}_{Day 24 , control}} ) \times 100$$

Here, V_{Day0,treatment} and V_{Day24,treatment} represent the tumor volumes of the treatment group on day 0 and day 24, respectively, and V_{Day0,control} and V_{Day24,control} represent the tumor volumes of the control group administered with PBS on day 0 and day 24, respectively.

### Experimental Example 1.13. Ki-67 and Caspase-3 Immunohistochemistry

Paraffin was removed from paraffin-embedded tumor sections (8 μm thick) according to a standard protocol. Heat-induced antigen retrieval was performed using a microwave with citrate buffer at pH 6.0 (Sigma-Aldrich, #C9999-100ML) placed in a Coplin jar. The antigen-retrieved tissue sections were permeabilized with 0.04% Triton X-100. To evaluate cell proliferation, the tissues were incubated with an anti-Ki67 monoclonal antibody (Santa Cruz; diluted 1:200) at 4° C. overnight, followed by incubation with an HRP-conjugated goat anti-mouse IgG (H+L) secondary antibody (Invitrogen, #31430; diluted 1:400) at room temperature for 2 hours. To evaluate cell death, the tissues were incubated with an anti-caspase-3/p17/p19 monoclonal antibody (Proteintech; diluted 1:100) at 4° C. overnight, followed by incubation with an HRP-conjugated goat anti-mouse IgG (H+L) secondary antibody (Invitrogen, #31430; diluted 1:400) at room temperature for 2 hours. Signals were developed by applying DAB. Hematoxylin staining was performed to distinguish nuclear components. The stained tissues were preserved by adding an organic mounting medium. All images were acquired using a BX51 optical microscope (Olympus).

### Experimental Example 1.14. TUNEL Assay

Whole tumor tissues were fixed in 10% neutral buffered formalin and then embedded in paraffin to prepare 8-μm-thick sections. Paraffin was removed from the paraffin-embedded tumor sections according to a standard protocol. Heat-induced antigen retrieval was performed using a microwave with citrate buffer at pH 6.0 (Sigma-Aldrich, #C9999-100ML) placed in a Coplin jar. Cell death was analyzed using a TUNEL Assay Kit-HRP-DAB (Abcam, #ab206386) according to the manufacturer's instructions. The DAB solution was used to detect the TdT-labeled reaction in the tumor tissues, thereby identifying DNA strand breaks generated during cell death. Hematoxylin counterstaining was performed to evaluate normal cells and apoptotic cells. An organic mounting medium was used for permanent preservation of the stained tissues. All images were acquired using a virtual microscope (Olympus).

### Experimental Example 2. Cell Death Experiment #1—Cell Death Effects of CRISPR/nCas Composition for Cell Death and PARP Inhibitor

### Experimental Example 2.1. Experimental Setup and Experimental Methods

To evaluate the cell death effects of a CRISPR/nCas composition for cell death and a PARP inhibitor, the following experiment was conducted. Specifically, Hela cells and HCT-116 cells cultured according to Experimental Example 1.1 were treated with a PARP inhibitor (olaparib) and a CRISPR/nCas composition for cell death having the compositions shown in the following table.

TABLE 1

| | | nCas | Scaffold of | Guide domain of | PARP |
|---|---|---|---|---|---|
| | Form | protein | guide RNAS | guide RNAs | inhibitor |
| Olaparib (−), | — | SEQ ID | No guide RNA | No guide RNA | No PARP |
| Mock | | NO: 2 | | | inhibitor |
| Olaparib (−), | RNP | SEQ ID | GUUUUAGAGCUAG | GCUCCGAUCGGGU | No PARP |
| NT | | NO: 2 | AAAUAGCAAGUUAA | UACAACA (SEQ ID | inhibitor |
| | | | AAUAAGGCUAGUC | NO: 53) | |
| | | | CGUUAUCAACUUG | | |
| | | | AAAAAGUGGCACC | | |
| | | | GAGUCGGUGCUUU | | |
| | | | UUU (SEQ ID | | |
| | | | NO: 8) | | |
| Olaparib (−), | RNP | SEQ ID | SEQ ID NO: 8 | CUAGAAUGACCAG | No PARP |
| Umix-4 | | NO: 2 | | UCAACAG (SEQ ID | inhibitor |
| | | | | NO: 31), | |
| | | | | GCUACCUAAGCAC | |
| | | | | AGCCACA (SEQ ID | |
| | | | | NO: 32), | |
| | | | | AUGGACCCCUAGG | |
| | | | | UCUCCGG (SEQ ID | |
| | | | | NO: 33), | |
| | | | | UGUUUGGUGGGUA | |
| | | | | CUCACCC (SEQ ID | |
| | | | | NO: 34) | |
| Olaparib (−), | RNP | SEQ ID | SEQ ID NO: 8 | AUAUUCCACAGGU | No PARP |
| Hmix-4 | | NO: 2 | | AUAUGCA (SEQ ID | inhibitor |
| | | | | NO: 35), | |
| | | | | GUUCAUGGCCAGA | |
| | | | | AGGACGA (SEQ ID | |
| | | | | NO: 36), | |
| | | | | UGGAAUGUGCCAC | |
| | | | | ACAGCAC (SEQ ID | |
| | | | | NO: 37), | |
| | | | | CUGCCUUGUCAUA | |
| | | | | GCUGUAC (SEQ ID | |
| | | | | NO: 38) | |
| Olaparib (−), | RNP | SEQ ID | SEQ ID NO: 8 | GCAGAGGCAGGUG | No PARP |
| MT-50 | | NO: 2 | | GAUCAUG (SEQ ID | inhibitor |
| | | | | NO: 39) | |
| Olaparib (+), | RNP | SEQ ID | No guide RNA | No guide RNA | Olaparib, |
| Mock | | NO: 2 | | | 1 μM |
| Olaparib (+), | RNP | SEQ ID | SEQ ID NO: 8 | SEQ ID NO: 53 | Olaparib, |
| NT | | NO: 2 | | | 1 μM |
| Olaparib (+), | RNP | SEQ ID | SEQ ID NO: 8 | SEQ ID NOs: 31 | Olaparib, |
| Umix-4 | | NO: 2 | | to 34 | 1 μM |
| Olaparib (+), | RNP | SEQ ID | SEQ ID NO: 8 | SEQ ID NOs: 35 | Olaparib, |
| Hmix-4 | | NO: 2 | | to 38 | 1 μM |
| Olaparib (+), | RNP | SEQ ID | SEQ ID NO: 8 | SEQ ID NO: 39 | Olaparib, |
| MT-50 | | NO: 2 | | | 1 μM |

In the table above, Umix-4 and Hmix-4 each comprised four types of guide RNAs according to the guide domain sequences shown in the table. For example, Umix-4 comprised four types of guide RNAs: 1) a guide RNA having a guide domain of SEQ ID NO: 31, 2) a guide RNA having a guide domain of SEQ ID NO: 32, 3) a guide RNA having a guide domain of SEQ ID NO: 33, and 4) a guide RNA having a guide domain of SEQ ID NO: 34. Each guide RNA had the same scaffold sequence of SEQ ID NO: 8. The same applies to all Experimental Examples described below.

The nCas protein used in this experiment was prepared according to Experimental Example 1.2, and the sgRNA used was prepared according to Experimental Example 1.3. The nCas protein and the sgRNA were administered to the cells in the form of RNPs according to Experimental Example 1.7. In the case of the PARP inhibitor, the concentrations disclosed in the above table were applied to each cell according to Experimental Example 1.6.

Thereafter, cell viability was measured according to Experimental Example 1.8.

### Experimental Example 2.2. Cell Death Experiment Results

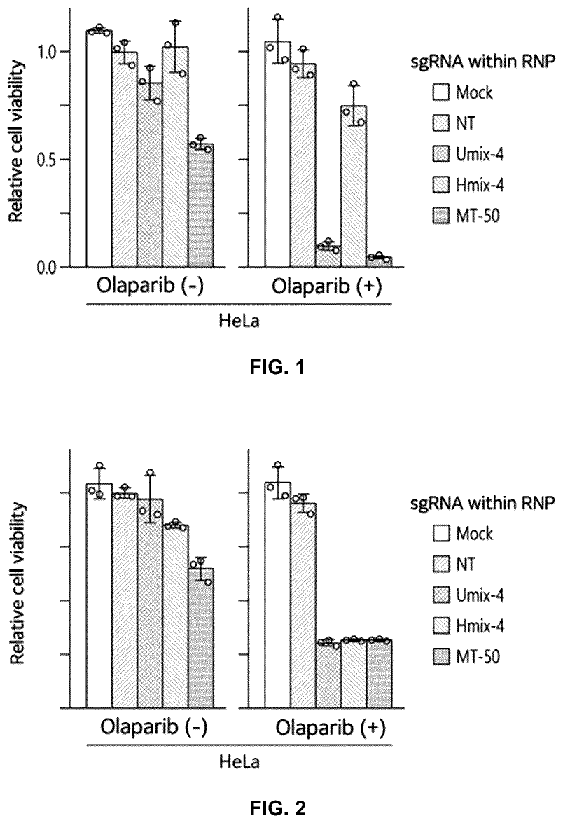

The experimental results according to Experimental Example 2.1 are shown in FIGS. 1 and 2. The quantitative data for FIGS. 1 and 2 are shown in the following tables.

TABLE 2

| | | | | |
|---|---|---|---|---|
| | Rep 1 | Rep 2 | Rep 3 | |
| RNP | (rel) | (rel) | (rel) | Average |

HeLa, Olaparib (−)

| | | | | |
|---|---|---|---|---|
| Mock | 1.115044 | 1.096304 | 1.088292 | 1.10 |
| NT | 1.043988 | 0.937012 | 1.019001 | 1.00 |
| Umix-4 | 0.862832 | 0.771473 | 0.928423 | 0.85 |
| Hmix-4 | 1.13706 | 0.900649 | 1.033056 | 1.02 |
| MT-50 | 0.601249 | 0.568454 | 0.54659 | 0.57 |

HeLa, Olaparib (+)

| | | | | |
|---|---|---|---|---|
| Mock | 1.162936 | 0.966995 | 1.020302 | 1.05 |
| NT | 0.893805 | 0.921395 | 1.017496 | 0.94 |
| Umix-4 | 0.079646 | 0.12025 | 0.096044 | 0.10 |
| Hmix-4 | 0.72228 | 0.673087 | 0.854243 | 0.75 |
| MT-50 | 0.04607 | 0.054659 | 0.038261 | 0.05 |

TABLE 3

| | | | | |
|---|---|---|---|---|
| | Rep 1 | Rep 2 | Rep 3 | |
| RNP | (rel) | (rel) | (rel) | Average |

HCT-116, Olaparib (−)

| | | | | |
|---|---|---|---|---|
| Mock | 1.011918 | 0.994637 | 1.126744 | 1.04 |
| NT | 0.98662 | 0.985567 | 1.027813 | 1.00 |
| Umix-4 | 0.900622 | 0.916543 | 1.099852 | 0.97 |
| Hmix-4 | 0.836431 | 0.868928 | 0.850019 | 0.85 |
| MT-50 | 0.687575 | 0.669142 | 0.58632 | 0.65 |

HCT-116, Olaparib (+)

| | | | | |
|---|---|---|---|---|
| Mock | 0.988918 | 1.026622 | 1.131285 | 1.05 |
| NT | 0.974643 | 0.981929 | 0.903936 | 0.95 |
| Umix-4 | 0.306751 | 0.317982 | 0.288419 | 0.30 |
| Hmix-4 | 0.316423 | 0.317562 | 0.3185 | 0.32 |
| MT-50 | 0.313691 | 0.318563 | 0.323116 | 0.32 |

The experimental results showed that, when 1) both the PARP inhibitor (olaparib) and the CRISPR/nCas composition for cell death were co-administered, and 2) a target nucleic acid was present in the genome of the cells, a high cell death effect was observed. On the other hand, when 1) the PARP inhibitor was not administered (Olaparib (−)), or 2) no target nucleic acid was present in the genome of the cells (the experimental group using the sgRNA of Hmix-4 in Hela cells), little to no cell death was observed. These results suggest that, when both the CRISPR/nCas composition for cell death and the PARP inhibitor are co-administered, only targeted cell death can be selectively induced with high efficiency. In addition, the results demonstrate that the cell death effect is significantly reduced when only a part of the above components is delivered or when non-target cells are treated.

### Experimental Example 3. Cell Death Experiment #2 Comparison of Effects of Administration of Nuclease, Nickase, and Nickase Together with PARP Inhibitor

### Experimental Example 3.1. Experimental Setup and Experimental Methods

To measure the cell death effects of a CRISPR/nCas composition for cell death and a PARP inhibitor, and to compare such effects with the cell death effects of a CRISPR/Cas composition having double-strand break activity, the following experiment was conducted. Specifically, Hela cells and HCT-116 cells cultured according to Experimental Example 1.1 were treated with a PARP inhibitor (olaparib) and a CRISPR/nCas composition for cell death having the compositions shown in the following table.

TABLE 4

| | sgRNA | | Cas/nCas | Scaffold of | Guide domain | PARP |
|---|---|---|---|---|---|---|
| Cas Label | Label | Form | protein | guide RNA | of guide RNA | inhibitor |
| Cas9^{WT} | Mock | RNP | SEQ ID | No guide | No guide | No PARP |
| | | | NO: 1 | RNA | RNA | inhibitor |
| Cas9^{WT} | NT | RNP | SEQ ID | SEQ ID | SEQ ID | No PARP |
| | | | NO: 1 | NO: 8 | NO: 53 | inhibitor |
| Cas9^{WT} | Umix-4 | RNP | SEQ ID | SEQ ID | SEQ ID NOs: | No PARP |
| | | | NO: 1 | NO: 8 | 31 to 34 | inhibitor |
| Cas9^{WT} | Hmix-4 | RNP | SEQ ID | SEQ ID | SEQ ID NOs: | No PARP |
| | | | NO: 1 | NO: 8 | 31 to 34 | inhibitor |
| Cas9^{WT} | MT-50 | RNP | SEQ ID | SEQ ID | SEQ ID | No PARP |
| | | | NO: 1 | NO: 8 | NO: 39 | inhibitor |
| nCas9^{D10A} | Mock | RNP | SEQ ID | No guide | No guide | No PARP |
| | | | NO: 2 | RNA | RNA | inhibitor |
| nCas9^{D10A} | NT | RNP | SEQ ID | SEQ ID | SEQ ID | No PARP |
| | | | NO: 2 | NO: 8 | NO: 53 | inhibitor |
| nCas9^{D10A} | Umix-4 | RNP | SEQ ID | SEQ ID | SEQ ID NOs: | No PARP |
| | | | NO: 2 | NO: 8 | 31 to 34 | inhibitor |
| nCas9^{D10A} | Hmix-4 | RNP | SEQ ID | SEQ ID | SEQ ID NOs: | No PARP |
| | | | NO: 2 | NO: 8 | 35 to 38 | inhibitor |
| nCas9^{D10A} | MT-50 | RNP | SEQ ID | SEQ ID | SEQ ID | No PARP |
| | | | NO: 2 | NO: 8 | NO: 39 | inhibitor |
| nCas9^{D10A} + | Mock | RNP | SEQ ID | No guide | No guide | Olaparib |
| Olaparib | | | NO: 2 | RNA | RNA | 1 μM |
| nCas9^{D10A} + | NT | RNP | SEQ ID | SEQ ID | SEQ ID | Olaparib |
| Olaparib | | | NO: 2 | NO: 8 | NO: 53 | 1 μM |
| nCas9^{D10A} + | Umix-4 | RNP | SEQ ID | SEQ ID | SEQ ID NOs: | Olaparib |
| Olaparib | | | NO: 2 | NO: 8 | 31 to 34 | 1 μM |
| nCas9^{D10A} + | Hmix-4 | RNP | SEQ ID | SEQ ID | SEQ ID NOs: | Olaparib |
| Olaparib | | | NO: 2 | NO: 8 | 35 to 38 | 1 μM |
| nCas9^{D10A} + | MT-50 | RNP | SEQ ID | SEQ ID | SEQ ID | Olaparib |
| Olaparib | | | NO: 2 | NO: 8 | NO: 39 | 1 μM |

The Cas protein and the nCas protein used in this experiment were prepared according to Experimental Example 1.2, and the sgRNA used was prepared according to Experimental Example 1.3. The nCas protein and the sgRNA were administered to the cells in the form of RNPs according to Experimental Example 1.7. In the case of the PARP inhibitor, the concentrations disclosed in the above table were applied to each cell according to Experimental Example 1.6.

Thereafter, each cell group was subjected to a colony formation assay according to Experimental Example 1.9.

### Experimental Example 3.2. Cell Death Experiment Results

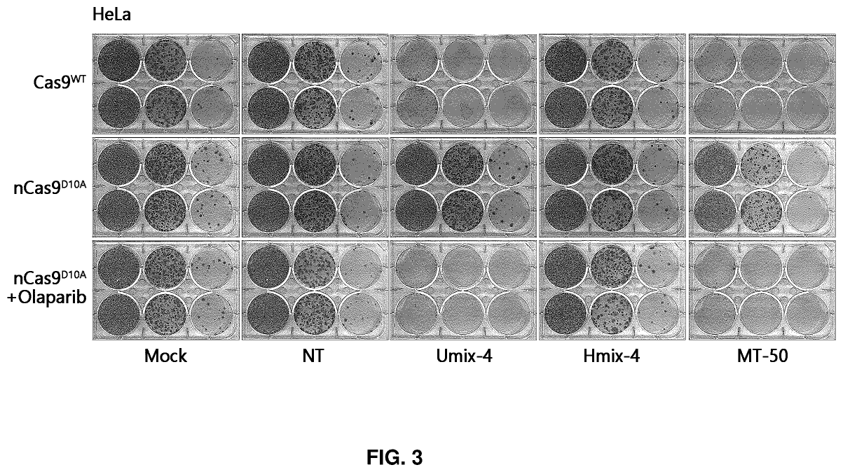

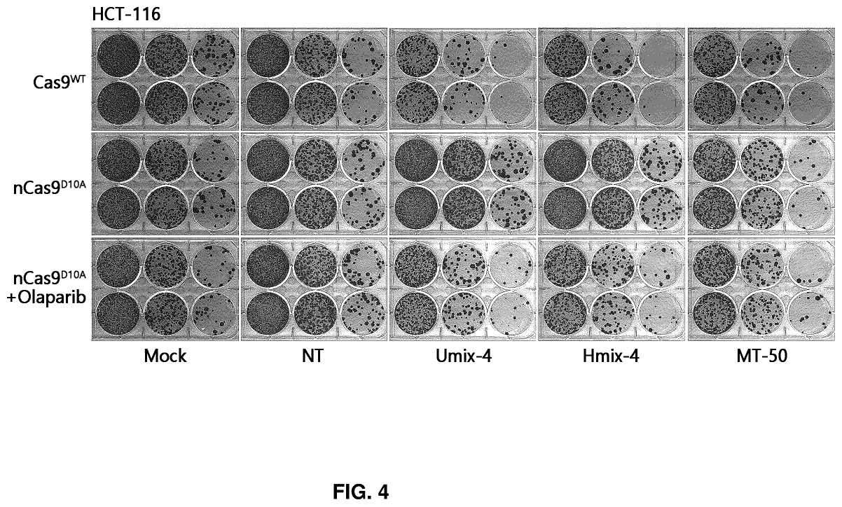

The experimental results according to Experimental Example 3.1 are shown in FIGS. 3 and 4.

The experimental results showed that, when 1) the CRISPR/Cas composition having double-strand break activity was administered, or both the PARP inhibitor (olaparib) and the CRISPR/nCas composition for cell death were co-administered, and 2) a target nucleic acid was present in the genome of the cells, a high cell death effect was observed. On the other hand, when 1) the PARP inhibitor was not administered (the experimental results of nCas9-D10A), or 2) no target nucleic acid was present in the genome of the cells (the experimental group using the sgRNA of Hmix-4 in Hela cells), little to no cell death was observed. As a result of this experiment, under conditions in which cell death is induced, a CRISPR/Cas composition having double-strand break activity (Cas9WT) exhibits a cell death effect equivalent to that of a CRISPR/nCas composition for cell death co-administered with a PARP inhibitor (nCas9-D10A+olaparib). In addition, the cell death effect is significantly reduced when only a part of the above components is delivered or when non-target cells are treated.

### Experimental Example 4. Cell Death Experiment #3-Comparison of Cell Death Effects Depending on Concentration of PARP Inhibitor

### Experimental Example 4.1. Experimental Setup and Experimental Methods

To compare cell death effects depending on the concentration of a PARP inhibitor administered, the following experiment was performed. Specifically, Hela cells and HCT-116 cells cultured according to Experimental Example 1.1 were treated with a PARP inhibitor (olaparib) and a CRISPR/nCas composition for cell death having the compositions shown in the following table.

TABLE 5

| | | nCas | Scaffold | Guide | |
|---|---|---|---|---|---|
| | | protein | of guide | domain of | Olaparib |
| Label | Form | or siRNA | RNA | guide RNA | concentration |
| Non- | — | — | — | — | 0.0001, 0.001, 0.01, |
| treated | | | | | 0.1, 1, or 10 μM |
| Umix-4 | RNP | SEQ ID | SEQ ID | SEQ ID NOS: | 0.0001, 0.001, 0.01, |
| | | NO: 2 | NO: 8 | 31 to 34 | 0.1, 1, or 10 μM |
| Hmix-4 | RNP | SEQ ID | SEQ ID | SEQ ID NOS: | 0.0001, 0.001, 0.01, |
| | | NO: 2 | NO: 8 | 35 to 38 | 0.1, 1, or 10 μM |
| SIBRC | SIRN | CUGGAAAAG | — | — | 0.0001, 0.001, 0.01, |
| A2 | A | GAAUACAGU | | | 0.1, 1, or 10 μM |
| | | UTT (SEQ | | | |
| | | ID NO: 40) | | | |

For each labeled material, experiments were conducted while varying the concentration of olaparib.

Here, in the case of siRNA, an siRNA having the sequence CUGGAAAAGGAAUACAGUUTT (SEQ ID NO: 40) was used, and 20 pmole thereof was delivered to the cells according to Experimental Example 1.7. After the experiment, cell viability was measured according to Experimental Example 1.8.

### Experimental Example 4.2. Cell Death Experiment Results

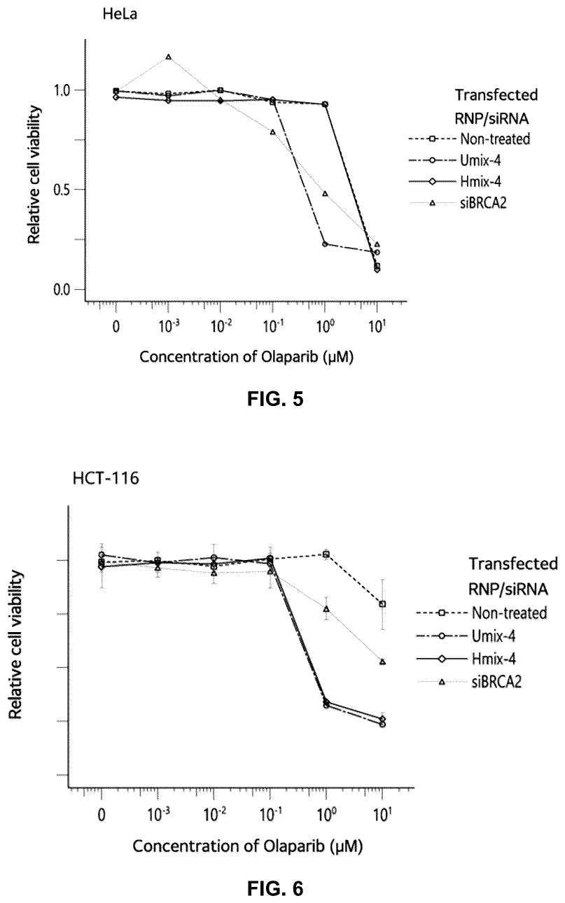

The experimental results according to Experimental Example 4.1 are shown in FIGS. 5 and 6. Additionally, the quantitative data for FIGS. 5 and 6 are shown in the following tables.

TABLE 6

| Cell | | Olaparib | Relative cell viability |
|---|---|---|---|
| Line | Label | Concentration | (average, n = 3) |
| HeLa | Non-treated | 0.0001 | 1.0000 |
| HeLa | Non-treated | 0.001 | 0.9810 |
| HeLa | Non-treated | 0.01 | 0.9959 |
| HeLa | Non-treated | 0.1 | 0.9391 |
| HeLa | Non-treated | 1 | 0.9279 |
| HeLa | Non-treated | 10 | 0.1192 |
| HeLa | siBRCA2 | 0.0001 | 0.9931 |
| HeLa | siBRCA2 | 0.001 | 1.1664 |
| HeLa | siBRCA2 | 0.01 | 0.9553 |
| HeLa | siBRCA2 | 0.1 | 0.7904 |
| HeLa | siBRCA2 | 1 | 0.4826 |
| HeLa | siBRCA2 | 10 | 0.2296 |
| HeLa | Umix-4 | 0.0001 | 0.9916 |
| HeLa | Umix-4 | 0.001 | 0.9689 |
| HeLa | Umix-4 | 0.01 | 0.9984 |
| HeLa | Umix-4 | 0.1 | 0.9542 |
| HeLa | Umix-4 | 1 | 0.2260 |
| HeLa | Umix-4 | 10 | 0.1860 |
| HeLa | Hmix-4 | 0.0001 | 0.9644 |
| HeLa | Hmix-4 | 0.001 | 0.9475 |
| HeLa | Hmix-4 | 0.01 | 0.9466 |
| HeLa | Hmix-4 | 0.1 | 0.9544 |
| HeLa | Hmix-4 | 1 | 0.9271 |
| HeLa | Hmix-4 | 10 | 0.1033 |

TABLE 7

| Cell | | Olaparib | Relative cell viability |
|---|---|---|---|
| Line | Label | Concentration | (average, n = 3) |
| HCT-116 | Non-treated | 0.0001 | 1.0000 |
| HCT-116 | Non-treated | 0.001 | 0.9939 |
| HCT-116 | Non-treated | 0.01 | 0.9743 |
| HCT-116 | Non-treated | 0.1 | 1.0072 |
| HCT-116 | Non-treated | 1 | 1.0307 |
| HCT-116 | Non-treated | 10 | 0.7979 |
| HCT-116 | siBRCA2 | 0.0001 | 0.9935 |
| HCT-116 | siBRCA2 | 0.001 | 0.9690 |
| HCT-116 | siBRCA2 | 0.01 | 0.9426 |
| HCT-116 | siBRCA2 | 0.1 | 0.9547 |
| HCT-116 | siBRCA2 | 1 | 0.7808 |
| HCT-116 | siBRCA2 | 10 | 0.5318 |
| HCT-116 | Umix-4 | 0.0001 | 1.0282 |
| HCT-116 | Umix-4 | 0.001 | 0.9932 |
| HCT-116 | Umix-4 | 0.01 | 1.0152 |
| HCT-116 | Umix-4 | 0.1 | 0.9870 |
| HCT-116 | Umix-4 | 1 | 0.3303 |
| HCT-116 | Umix-4 | 10 | 0.2380 |
| HCT-116 | Hmix-4 | 0.0001 | 0.9711 |
| HCT-116 | Hmix-4 | 0.001 | 0.9927 |
| HCT-116 | Hmix-4 | 0.01 | 0.9849 |
| HCT-116 | Hmix-4 | 0.1 | 1.0178 |
| HCT-116 | Hmix-4 | 1 | 0.3398 |
| HCT-116 | Hmix-4 | 10 | 0.2630 |

The experimental results showed that when both the PARP inhibitor (olaparib) and the CRISPR/nCas composition for cell death were co-administered, cell death effects were observed even at a concentration (1 μM) that is one-tenth of the concentration (10 μM) at which the PARP inhibitor exhibits cytotoxicity. This suggests that, when both the PARP inhibitor and the CRISPR/nCas composition for cell death are co-administered, the PARP inhibitor can be administered at a concentration much lower than the concentration at which cytotoxicity is exhibited when administered alone, thereby indicating reduced side effects and improved safety.

### Experimental Example 5. Cell Death Experiment #4-Cell Death Effects Depending on Type of PARP Inhibitor

### Experimental Example 5.1. Experimental Setup and Experimental Methods

To confirm cell death effects depending on the type of PARP inhibitor, the following experiment was performed. Specifically, Hela cells and HCT-116 cells cultured according to Experimental Example 1.1 were treated with a PARP inhibitor and a CRISPR/nCas composition for cell death having the compositions shown in the following table.

TABLE 8

| | | | | | | PARP inhibitor |
|---|---|---|---|---|---|---|
| | sgRNA | | Cas/nCas | Scaffold of | Guide domain | (or another |
| Cas label | label | Form | protein | guide RNA | of guide RNA | agent) |
| Cas9^{WT} | Mock | — | SEQ ID | No guide | No guide | DMSO |
| | | | NO: 1 | RNA | RNA | |
| Cas9^{WT} | NT | RNP | SEQ ID | SEQ ID | SEQ ID | DMSO |
| | | | NO: 1 | NO: 8 | NO: 53 | |
| Cas9^{WT} | Umix-2 | RNP | SEQ ID | SEQ ID | SEQ ID NOS: | DMSO |
| | | | NO: 1 | NO: 8 | 31 to 32 | |
| Cas9^{WT} | Umix-3 | RNP | SEQ ID | SEQ ID | SEQ ID NOS: | DMSO |
| | | | NO: 1 | NO: 8 | 31 to 33 | |
| Cas9^{WT} | Umix-4 | RNP | SEQ ID | SEQ ID | SEQ ID NOS: | DMSO |
| | | | NO: 1 | NO: 8 | 31 to 34 | |
| Cas9^{WT} | MT-50 | RNP | SEQ ID | SEQ ID | SEQ ID | DMSO |
| | | | NO: 1 | NO: 8 | NO: 39 | |
| nCas9^{D10A} | Mock | — | SEQ ID | No guide | No guide | One of DMSO, Olaparib, |
| | | | NO: 2 | RNA | RNA | Rucaparib, Niraparib, |
| | | | | | | Velaparib, or Iniparib |
| nCas9^{D10A} | NT | RNP | SEQ ID | SEQ ID | SEQ ID | One of DMSO, Olaparib, |
| | | | NO: 2 | NO: 8 | NO: 53 | Rucaparib, Niraparib, |
| | | | | | | Velaparib, or Iniparib |
| nCas9^{D10A} | Drug | — | No Cas | No guide | No guide | One of DMSO, Olaparib, |
| | only | | protein | RNA | RNA | Rucaparib, Niraparib, |
| | | | | | | Velaparib, or Iniparib |
| nCas9^{D10A} | Umix-2 | RNP | SEQ ID | SEQ ID | SEQ ID NOS: | One of DMSO, Olaparib, |
| | | | NO: 2 | NO: 8 | 31 to 32 | Rucaparib, Niraparib, |
| | | | | | | Velaparib, or Iniparib |
| nCas9^{D10A} | Umix-3 | RNP | SEQ ID | SEQ ID | SEQ ID NOS: | One of DMSO, Olaparib, |
| | | | NO: 2 | NO: 8 | 31 to 33 | Rucaparib, Niraparib, |
| | | | | | | Velaparib, or Iniparib |
| nCas9^{D10A} | Umix-4 | RNP | SEQ ID | SEQ ID | SEQ ID NOS: | One of DMSO, Olaparib, |
| | | | NO: 2 | NO: 8 | 31 to 34 | Rucaparib, Niraparib, |
| | | | | | | Velaparib, or Iniparib |
| nCas9^{D10A} | MT-50 | RNP | SEQ ID | SEQ ID | SEQ ID | One of DMSO, Olaparib, |
| | | | NO: 2 | NO: 8 | NO: 39 | Rucaparib, Niraparib, |
| | | | | | | Velaparib, or Iniparib |

For each RNP composition of the embodiment using nCas9^{D10A}, one PARP inhibitor selected from olaparib, rucaparib, niraparib, veliparib, and iniparib was co-administered. DMSO was used as a control.

The Cas protein and the nCas protein used in this experiment were prepared according to Experimental Example 1.2, and the sgRNA used was prepared according to Experimental Example 1.3. The nCas protein and the sgRNA were administered to the cells in the form of RNPs according to Experimental Example 1.7. The cells were each treated with the respective PARP inhibitors at the concentrations disclosed in Experimental Example 1.6 using the materials disclosed in the above table.

Thereafter, cell viability was measured according to Experimental Example 1.8.

### Experimental Example 5.2. Cell Death Experiment Results Depending on Type of PARP Inhibitor

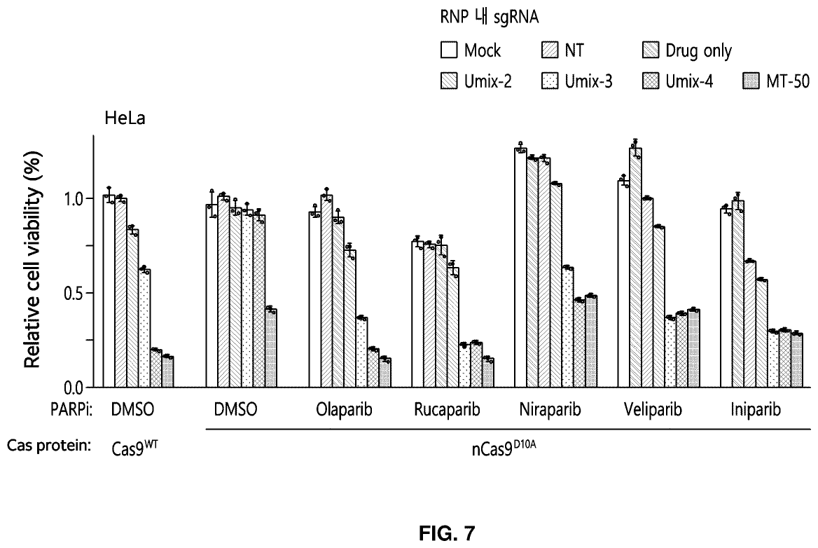

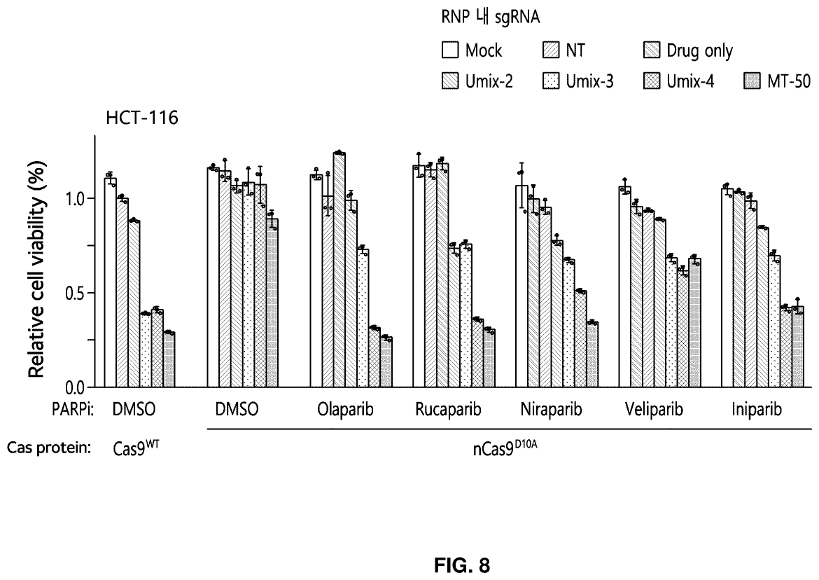

The experimental results according to Experimental Example 5.1 are shown in FIGS. 7 and 8. The quantitative data for FIGS. 7 and 8 are shown in the following tables.

TABLE 9

| | | | | Relative cell viability |
|---|---|---|---|---|
| CellLine | CRISPR | PARPi | Target | (average, n = 3) |
| HeLa | Cas9^{WT} | DMSO | Mock | 1 |
| HeLa | Cas9^{WT} | DMSO | Non-target | 0.9838 |
| HeLa | Cas9^{WT} | DMSO | Umix-2 | 0.8224 |
| HeLa | Cas9^{WT} | DMSO | Umix-3 | 0.6143 |
| HeLa | Cas9^{WT} | DMSO | Umix-4 | 0.1972 |
| HeLa | Cas9^{WT} | DMSO | MT-50 | 0.1610 |
| HeLa | nCas9^{D10A} | DMSO | Mock | 0.9493 |
| HeLa | nCas9^{D10A} | DMSO | Non-target | 0.9947 |
| HeLa | nCas9^{D10A} | DMSO | Umix-2 | 0.9348 |
| HeLa | nCas9^{D10A} | DMSO | Umix-3 | 0.9250 |
| HeLa | nCas9^{D10A} | DMSO | Umix-4 | 0.8973 |
| HeLa | nCas9^{D10A} | DMSO | MT-50 | 0.4079 |
| HeLa | nCas9^{D10A} | Olaparib | Mock | 0.9135 |
| HeLa | nCas9^{D10A} | Olaparib | Non-target | 1.0018 |
| HeLa | nCas9^{D10A} | Olaparib | Umix-2 | 0.7135 |
| HeLa | nCas9^{D10A} | Olaparib | Umix-3 | 0.3623 |
| HeLa | nCas9^{D10A} | Olaparib | Umix-4 | 0.1999 |
| HeLa | nCas9^{D10A} | Olaparib | MT-50 | 0.1507 |
| HeLa | nCas9^{D10A} | Olaparib | drug_only | 0.8856 |
| HeLa | nCas9^{D10A} | Rucaparib | Mock | 0.7584 |
| HeLa | nCas9^{D10A} | Rucaparib | Non-target | 0.7436 |
| HeLa | nCas9^{D10A} | Rucaparib | Umix-2 | 0.6221 |
| HeLa | nCas9^{D10A} | Rucaparib | Umix-3 | 0.2234 |
| HeLa | nCas9^{D10A} | Rucaparib | Umix-4 | 0.2354 |
| HeLa | nCas9^{D10A} | Rucaparib | MT-50 | 0.1482 |
| HeLa | nCas9^{D10A} | Rucaparib | drug_only | 0.7407 |
| HeLa | nCas9^{D10A} | Niraparib | Mock | 1.2444 |
| HeLa | nCas9^{D10A} | Niraparib | Non-target | 1.1930 |
| HeLa | nCas9^{D10A} | Niraparib | Umix-2 | 1.0610 |
| HeLa | nCas9^{D10A} | Niraparib | Umix-3 | 0.6240 |
| HeLa | nCas9^{D10A} | Niraparib | Umix-4 | 0.4544 |
| HeLa | nCas9^{D10A} | Niraparib | MT-50 | 0.4796 |
| HeLa | nCas9^{D10A} | Niraparib | drug_only | 1.1924 |
| HeLa | nCas9^{D10A} | Veliparib | Mock | 1.0773 |
| HeLa | nCas9^{D10A} | Veliparib | Non-target | 0.9843 |
| HeLa | nCas9^{D10A} | Veliparib | Umix-2 | 0.8355 |
| HeLa | nCas9^{D10A} | Veliparib | Umix-3 | 0.3616 |
| HeLa | nCas9^{D10A} | Veliparib | Umix-4 | 0.3864 |
| HeLa | nCas9^{D10A} | Veliparib | MT-50 | 0.4051 |
| HeLa | nCas9^{D10A} | Veliparib | drug_only | 1.2443 |

TABLE 10

| | | | | Relative cell viability |
|---|---|---|---|---|
| CellLine | CRISPR | PARPi | Target | (average, n = 3) |
| HCT-116 | Cas9^{WT} | DMSO | Mock | 1 |
| HCT-116 | Cas9^{WT} | DMSO | Non-target | 0.9038 |
| HCT-116 | Cas9^{WT} | DMSO | Umix-2 | 0.7973 |
| HCT-116 | Cas9^{WT} | DMSO | Umix-3 | 0.3527 |
| HCT-116 | Cas9^{WT} | DMSO | Umix-4 | 0.3706 |
| HCT-116 | Cas9^{WT} | DMSO | MT-50 | 0.2636 |
| HCT-116 | nCas9^{D10A} | DMSO | Mock | 1.0499 |
| HCT-116 | nCas9^{D10A} | DMSO | Non-target | 1.0337 |
| HCT-116 | nCas9^{D10A} | DMSO | Umix-2 | 0.9629 |
| HCT-116 | nCas9^{D10A} | DMSO | Umix-3 | 0.9809 |
| HCT-116 | nCas9^{D10A} | DMSO | Umix-4 | 0.9679 |
| HCT-116 | nCas9^{D10A} | DMSO | MT-50 | 0.8059 |
| HCT-116 | nCas9^{D10A} | Olaparib | Mock | 1.0164 |
| HCT-116 | nCas9^{D10A} | Olaparib | Non-target | 0.9147 |
| HCT-116 | nCas9^{D10A} | Olaparib | Umix-2 | 0.8940 |
| HCT-116 | nCas9^{D10A} | Olaparib | Umix-3 | 0.6589 |
| HCT-116 | nCas9^{D10A} | Olaparib | Umix-4 | 0.2872 |
| HCT-116 | nCas9^{D10A} | Olaparib | MT-50 | 0.2388 |
| HCT-116 | nCas9^{D10A} | Olaparib | drug_only | 1.1219 |
| HCT-116 | nCas9^{D10A} | Rucaparib | Mock | 1.0585 |
| HCT-116 | nCas9^{D10A} | Rucaparib | Non-target | 1.0405 |
| HCT-116 | nCas9^{D10A} | Rucaparib | Umix-2 | 0.6658 |
| HCT-116 | nCas9^{D10A} | Rucaparib | Umix-3 | 0.6846 |
| HCT-116 | nCas9^{D10A} | Rucaparib | Umix-4 | 0.3262 |
| HCT-116 | nCas9^{D10A} | Rucaparib | MT-50 | 0.2782 |
| HCT-116 | nCas9^{D10A} | Rucaparib | drug_only | 1.0701 |
| HCT-116 | nCas9^{D10A} | Niraparib | Mock | 0.9651 |
| HCT-116 | nCas9^{D10A} | Niraparib | Non-target | 0.8608 |
| HCT-116 | nCas9^{D10A} | Niraparib | Umix-2 | 0.7023 |
| HCT-116 | nCas9^{D10A} | Niraparib | Umix-3 | 0.6104 |
| HCT-116 | nCas9^{D10A} | Niraparib | Umix-4 | 0.4612 |
| HCT-116 | nCas9^{D10A} | Niraparib | MT-50 | 0.3127 |
| HCT-116 | nCas9^{D10A} | Niraparib | drug_only | 0.9015 |
| HCT-116 | nCas9^{D10A} | Veliparib | Mock | 0.9574 |
| HCT-116 | nCas9^{D10A} | Veliparib | Non-target | 0.8435 |
| HCT-116 | nCas9^{D10A} | Veliparib | Umix-2 | 0.8020 |
| HCT-116 | nCas9^{D10A} | Veliparib | Umix-3 | 0.6189 |
| HCT-116 | nCas9^{D10A} | Veliparib | Umix-4 | 0.5577 |
| HCT-116 | nCas9^{D10A} | Veliparib | MT-50 | 0.6136 |
| HCT-116 | nCas9^{D10A} | Veliparib | drug_only | 0.8648 |

The experimental results showed that, when each PARP inhibitor and the CRISPR/nCas composition for cell death were administered, a cell death effect at or above a certain level was observed in all cases where 1) both the PARP inhibitor and the CRISPR/nCas composition for cell death were delivered, and 2) three or more target nucleic acids were present. This demonstrates that, regardless of the type of PARP inhibitor, when 1) a PARP-mediated DNA repair mechanism is inhibited and 2) single-strand breaks are induced at three or more sites within the genome of cells by a CRISPR/nCas composition, the cells undergo cell death. Furthermore, considering that little to no cell death effect was observed in embodiments in which any type of PARP inhibitor was administered alone, it can be seen that cells can be induced to undergo cell death by combination with a CRISPR/nCas composition for cell death, even when a PARP inhibitor is used at a concentration exhibiting little to no cytotoxicity.

### Experimental Example 6. Confirmation of Whether Mutations are Introduced Upon Administration of CRISPR/nCas Composition and PARP Inhibitor

### Experimental Example 6.1. Experimental Setup and Experimental Methods

To compare the extent to which mutations are introduced into a genome when cells are administered with 1) a CRISPR/Cas composition having double-strand break activity, 2) a CRISPR/nCas composition, and 3) a CRISPR/nCas composition together with a PARP inhibitor, the following experiment was performed. Specifically, Hela cells and HCT-116 cells cultured according to Experimental Example 1.1 were treated with materials having the compositions shown in the following table.

TABLE 11

| RNP | Target | Form | Cas/nCas | Scaffold of | Guide domain |
|---|---|---|---|---|---|
| transfected | Gene | | protein | guide RNA | of guide RNA |
| Cas9WT | HPRT1 | RNP | SEQ ID | SEQ ID | CUAGAAUGACCAGUCAA |
| | | | NO: 1 | NO: 8 | CAG (SEQ ID NO: 41) |
| Cas9WT | BCR | RNP | SEQ ID | SEQ ID | GCUACCUAAGCACAGCC |
| | | | NO: 1 | NO: 8 | ACA (SEQ ID NO: 42) |
| Cas9WT | DNAJC6 | RNP | SEQ ID | SEQ ID | AUGGACCCCUAGGUCUC |
| | | | NO: 1 | NO: 8 | CGG (SEQ ID NO: 43) |
| Cas9WT | PLXDC2 | RNP | SEQ ID | SEQ ID | UGUUUGGUGGGUACUCA |
| | | | NO: 1 | NO: 8 | CCC (SEQ ID NO: 44) |
| nCas9D10A | HPRT1 | RNP | SEQ ID | SEQ ID | SEQ ID NO: 41 |
| | | | NO: 2 | NO: 8 | |
| nCas9D10A | BCR | RNP | SEQ ID | SEQ ID | SEQ ID NO: 42 |
| | | | NO: 2 | NO: 8 | |
| nCas9D10A | DNAJC6 | RNP | SEQ ID | SEQ ID | SEQ ID NO: 43 |
| | | | NO: 2 | NO: 8 | |
| nCas9D10A | PLXDC2 | RNP | SEQ ID | SEQ ID | SEQ ID NO: 44 |
| | | | NO: 2 | NO: 8 | |
| nCas9D10A + | HPRT1 | RNP | SEQ ID | SEQ ID | SEQ ID NO: 41 |
| Olaparib | | | NO: 2 | NO: 8 | |
| nCas9D10A + | BCR | RNP | SEQ ID | SEQ ID | SEQ ID NO: 42 |
| Olaparib | | | NO: 2 | NO: 8 | |
| nCas9D10A + | DNAJC6 | RNP | SEQ ID | SEQ ID | SEQ ID NO: 43 |
| Olaparib | | | NO: 2 | NO: 8 | |
| nCas9D10A + | PLXDC2 | RNP | SEQ ID | SEQ ID | SEQ ID NO: 44 |
| Olaparib | | | NO: 2 | NO: 8 | |

The Cas protein and the nCas protein used in this experiment were prepared according to Experimental Example 1.2, and the sgRNA used was prepared according to Experimental Example 1.3. The nCas protein and the sgRNA were administered to the cells in the form of RNPs according to Experimental Example 1.7. The cells were each treated with the respective PARP inhibitors at the concentrations disclosed in the above table according to Experimental Example 1.6.

Thereafter, sequencing was performed according to Experimental Example 1.10 to confirm whether indels were introduced. The primers used for sequencing are shown in the following table.

TABLE 12

| Label | DNA Sequence (5′ to 3′) |
|---|---|
| HPRT1- | TCGTCGGCAGCGTCAGATGTGTATAAGAGACAGGTG |
| Forward | TACATAAGGATATACATATACA (SEQ ID NO: 45) |
| HPRT1- | GTCTCGTGGGCTCGGAGATGTGTATAAGAGACAGCA |
| Backward | TACCTTTCCAGTTAAAGTT (SEQ ID NO: 46) |
| BCR- | TCGTCGGCAGCGTCAGATGTGTATAAGAGACAGGAG |
| Forward | CCATGCTGCTGTCTGTA (SEQ ID NO: 47) |
| BCR- | GTCTCGTGGGCTCGGAGATGTGTATAAGAGACAGAG |
| Backward | GCCTGGAGGAGTCTTAGC (SEQ ID NO: 48) |
| DNAJC6- | TCGTCGGCAGCGTCAGATGTGTATAAGAGACAGCTG |
| Forward | GAGGATTCTGGGGTTTT (SEQ ID NO: 49) |
| DNAJC6- | GTCTCGTGGGCTCGGAGATGTGTATAAGAGACAGAG |
| Backward | CGCAATAGGAAGGAAGG (SEQ ID NO: 50) |
| PLXDC2- | TCGTCGGCAGCGTCAGATGTGTATAAGAGACAGAAA |
| Forward | AGAGGGCAGAAGGATGG (SEQ ID NO: 51) |
| PLXDC2- | GTCTCGTGGGCTCGGAGATGTGTATAAGAGACAGAA |
| Backward | TTGCCCAGAAAACACAGG (SEQ ID NO: 52) |

### Experimental Example 6.2. Experimental Results

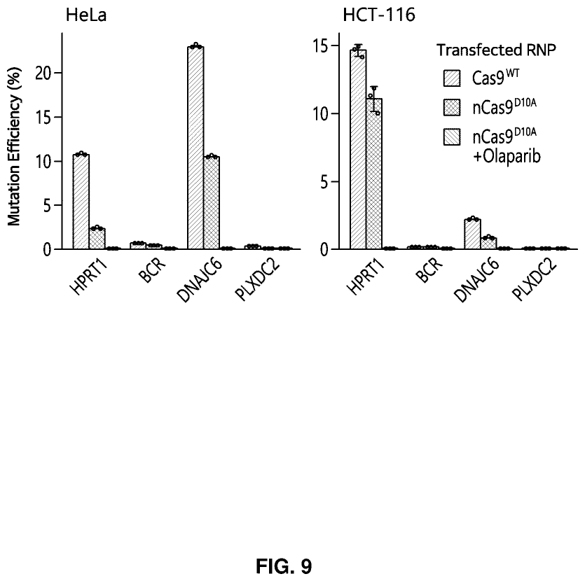

The experimental results according to Experimental Example 6.1 are shown in FIG. 9. The quantitative data for FIG. 9 are shown in the following table.

TABLE 13

| | | | Mutation Efficiency |
|---|---|---|---|
| CellLine | CRISPR | Target | (%, Average, n = 3) |
| HeLa | Cas9^{WT} | HPRT1 | 10.79 |
| HeLa | Cas9^{WT} | BCR | 0.74 |
| HeLa | Cas9^{WT} | DNAJC6 | 22.96 |
| HeLa | Cas9^{WT} | PLXDC2 | 0.42 |
| HeLa | nCas9^{D10A} | HPRT1 | 2.40 |
| HeLa | nCas9^{D10A} | BCR | 0.50 |
| HeLa | nCas9^{D10A} | DNAJC6 | 10.52 |
| HeLa | nCas9^{D10A} | PLXDC2 | 0.12 |
| HeLa | nCas9^{D10A} + | HPRT1 | 0.17 |
| | Olaparib | | |
| HeLa | nCas9^{D10A} + | BCR | 0.06 |
| | Olaparib | | |
| HeLa | nCas9^{D10A} + | DNAJC6 | 0.19 |
| | Olaparib | | |
| HeLa | nCas9^{D10A} + | PLXDC2 | 0.05 |
| | Olaparib | | |
| HCT-116 | Cas9^{WT} | HPRT1 | 14.63 |
| HCT-116 | Cas9^{WT} | BCR | 0.19 |
| HCT-116 | Cas9^{WT} | DNAJC6 | 2.22 |
| HCT-116 | Cas9^{WT} | PLXDC2 | 0.06 |
| HCT-116 | nCas9^{D10A} | HPRT1 | 11.08 |
| HCT-116 | nCas9^{D10A} | BCR | 0.20 |
| HCT-116 | nCas9^{D10A} | DNAJC6 | 0.83 |
| HCT-116 | nCas9^{D10A} | PLXDC2 | 0.08 |
| HCT-116 | nCas9^{D10A} + | HPRT1 | 0.14 |
| | Olaparib | | |
| HCT-116 | nCas9^{D10A} + | BCR | 0.06 |
| | Olaparib | | |
| HCT-116 | nCas9^{D10A} + | DNAJC6 | 0.07 |
| | Olaparib | | |
| HCT-116 | nCas9^{D10A} + | PLXDC2 | 0.08 |
| | Olaparib | | |

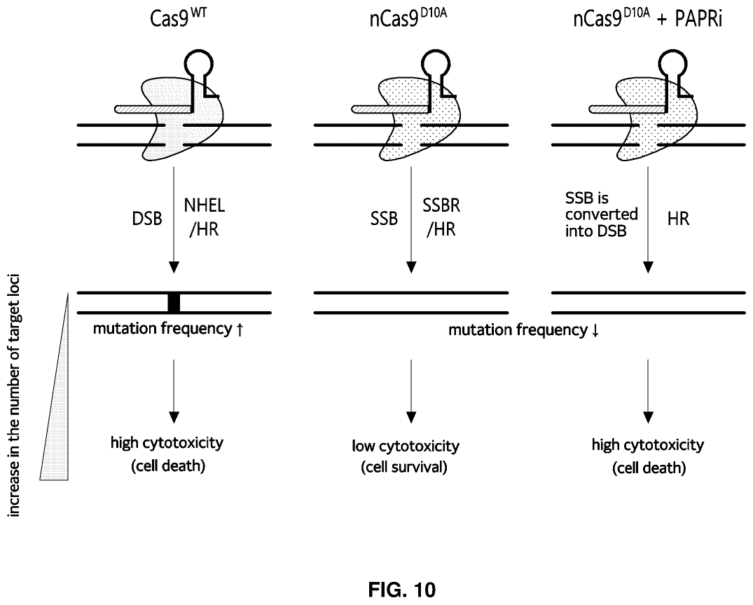

The experimental results showed that the CRISPR/Cas composition having double-strand break activity was observed to induce the highest level of mutations, followed by the CRISPR/nCas composition, whereas little to no mutations were introduced when the CRISPR/nCas composition and the PARP inhibitor were administered. This can be explained by a reduction in the frequency of mutation introduction because, when both a CRISPR/nCas composition and a PARP inhibitor are co-administered, 1) single-strand breaks generated by the CRISPR/nCas composition are converted into double-strand breaks, and 2) such damage is repaired through a homologous recombination (HR) repair mechanism. An illustration of this mechanism is shown in FIG. 10. Additionally, the above experimental results also suggest that single-strand breaks generated by the CRISPR/nCas composition are converted into double-strand breaks. Accordingly, it can be presumed that, when a CRISPR/nCas composition and a PARP inhibitor are co-administered to induce cell death, the underlying cause is the conversion of a plurality of (that is, three or more) single-strand breaks within the genome of cells into double-strand breaks.

### Experimental Example 7. Tumor Growth Inhibition Experiment in HCT-116 Tumor Xenograft Mouse Model

The tumor xenograft mouse model prepared according to Experimental Example 1.10 was randomly divided into four groups. Then, a PARP inhibitor and a CRISPR/nCas composition for cell death were administered according to Experimental Example 1.11 to confirm tumor growth inhibition effects. Specifically, 28.5 μg of olaparib diluted in PBS and the CRISPR/nCas composition for cell death were administered. The materials administered to each group are shown in the following table.

TABLE 14

| | | nCas | Scaffold of | Guide domain | PARP |
|---|---|---|---|---|---|
| Label | Form | protein | guide RNA | of guide RNA | inhibitor |
| PBS | — | — | — | — | — |
| Olaparib | — | — | — | — | Olaparib |
| only | | | | | 28.5 μg |
| RNP | RNP | SEQ ID | SEQ ID | SEQ ID NOs: | — |
| (Hmix-4) | | NO: 2 | NO: 8 | 35 to 38 | |
| only | | | | | |
| RNP | RNP | SEQ ID | SEQ ID | SEQ ID NOs: | Olaparib |
| (Hmix-4) + | | NO: 2 | NO: 8 | 35 to 38 | 28.5 μg |
| Olaparib | | | | | |

Thereafter, immunostaining for Ki67 and caspase-3 was performed according to Experimental Example 1.13, and TUNEL histopathological analysis was performed according to Experimental Example 1.14.

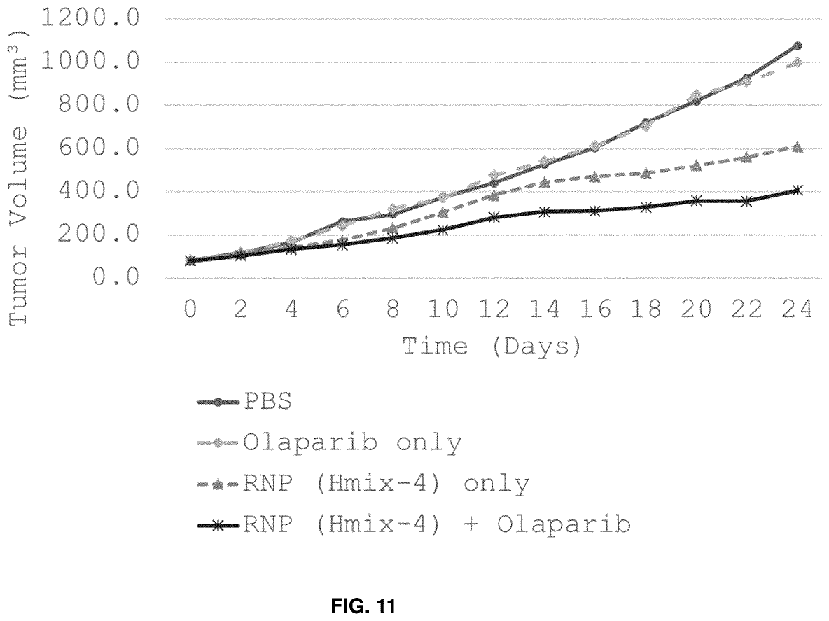

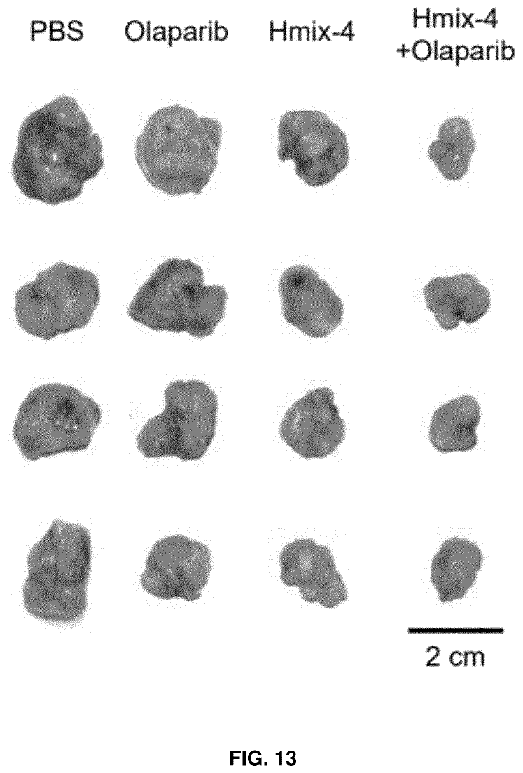

The experimental results are shown in the following table and FIGS. 11 to 13.

TABLE 15

| | | Volume of Tumor | Standard |
|---|---|---|---|
| RNP | Day | (mm³, Average, n = 4) | Deviation |
| PBS | 0 | 80.4 | 10.39 |
| PBS | 2 | 115.1 | 20.13 |
| PBS | 4 | 166.5 | 14.04 |
| PBS | 6 | 262.1 | 56.75 |
| PBS | 8 | 295.0 | 35.49 |
| PBS | 10 | 375.5 | 19.19 |
| PBS | 12 | 439.3 | 63.37 |
| PBS | 14 | 525.7 | 111.00 |
| PBS | 16 | 603.3 | 156.96 |
| PBS | 18 | 720.1 | 276.37 |
| PBS | 20 | 817.8 | 292.37 |
| PBS | 22 | 925.5 | 291.58 |
| PBS | 24 | 1075.6 | 357.24 |
| Olaparib only | 0 | 82.1 | 9.96 |
| Olaparib only | 2 | 117.0 | 20.33 |
| Olaparib only | 4 | 172.6 | 16.94 |
| Olaparib only | 6 | 241.8 | 53.08 |
| Olaparib only | 8 | 320.3 | 41.45 |
| Olaparib only | 10 | 371.2 | 12.62 |
| Olaparib only | 12 | 476.0 | 48.06 |
| Olaparib only | 14 | 542.7 | 86.33 |
| Olaparib only | 16 | 610.7 | 134.58 |
| Olaparib only | 18 | 702.9 | 238.54 |
| Olaparib only | 20 | 847.8 | 252.78 |
| Olaparib only | 22 | 907.4 | 262.77 |
| Olaparib only | 24 | 996.7 | 318.71 |
| RNP (Hmix-4) only | 0 | 83.3 | 9.51 |
| RNP (Hmix-4) only | 2 | 116.3 | 24.49 |
| RNP (Hmix-4) only | 4 | 141.2 | 18.31 |
| RNP (Hmix-4) only | 6 | 175.6 | 47.83 |
| RNP (Hmix-4) only | 8 | 233.2 | 36.15 |
| RNP (Hmix-4) only | 10 | 306.0 | 11.75 |
| RNP (Hmix-4) only | 12 | 383.8 | 27.26 |
| RNP (Hmix-4) only | 14 | 443.8 | 72.23 |
| RNP (Hmix-4) only | 16 | 470.1 | 125.88 |
| RNP (Hmix-4) only | 18 | 486.6 | 219.26 |
| RNP (Hmix-4) only | 20 | 520.7 | 247.54 |
| RNP (Hmix-4) only | 22 | 559.7 | 239.88 |
| RNP (Hmix-4) only | 24 | 609.1 | 207.15 |
| RNP (Hmix-4) + | 0 | 79.3 | 6.28 |
| Olaparib | | | |
| RNP (Hmix-4) + | 2 | 103.8 | 27.79 |
| Olaparib | | | |
| RNP (Hmix-4) + | 4 | 133.9 | 19.41 |
| Olaparib | | | |
| RNP (Hmix-4) + | 6 | 153.4 | 31.27 |
| Olaparib | | | |
| RNP (Hmix-4) + | 8 | 184.9 | 38.04 |
| Olaparib | | | |
| RNP (Hmix-4) + | 10 | 223.0 | 13.37 |
| Olaparib | | | |
| RNP (Hmix-4) + | 12 | 280.9 | 33.36 |
| Olaparib | | | |
| RNP (Hmix-4) + | 14 | 306.5 | 75.11 |
| Olaparib | | | |
| RNP (Hmix-4) + | 16 | 311.2 | 128.59 |
| Olaparib | | | |
| RNP (Hmix-4) + | 18 | 328.7 | 185.91 |
| Olaparib | | | |
| RNP (Hmix-4) + | 20 | 357.3 | 221.87 |
| Olaparib | | | |
| RNP (Hmix-4) + | 22 | 356.5 | 203.91 |
| Olaparib | | | |
| RNP (Hmix-4) + | 24 | 405.6 | 151.13 |
| Olaparib | | | |

The results of tumor growth inhibition rates calculated according to Experimental Example 1.12 are shown in the following table.

TABLE 16

| RNP | Tumor Inhibition Rate (%) |
|---|---|
| Olaparib_only | 8.10% |
| Hmix-4_only | 47.16% |
| Hmix-4 + | 67.21% |
| Olaparib | |

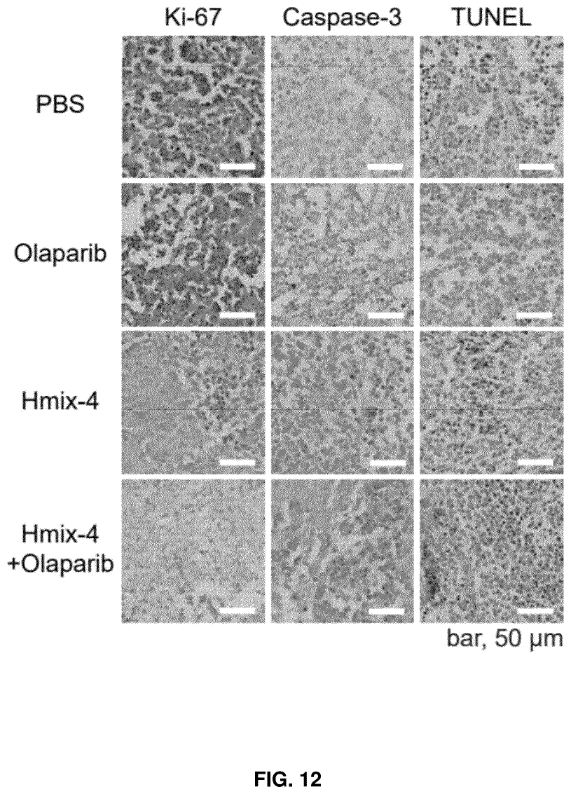

The experimental results showed that tumor growth was efficiently inhibited when treated with the PARP inhibitor (olaparib) together with the CRISPR/nCas composition for cell death (nCas^{D10A}). Furthermore, tumor growth inhibition by the above combination occurred without any observable side effects or body weight loss, suggesting selective induction of cell death only in tumor cells. Additionally, increased levels of cell death (caspase-3-positive and TUNEL-positive cells) and decreased levels of proliferating cells (Ki67-positive cells) were observed (FIG. 12), indicating that tumor growth inhibition was induced by DNA damage and subsequent cell death.

### INDUSTRIAL APPLICABILITY

The cell death method disclosed herein can selectively induce cell death only in target cells. In addition, undesired side effects, such as the introduction of mutations into non-target cells, can be minimized. Accordingly, the cell death method can be utilized to induce the death of specific cell populations that are undesirable to patients, for example, to selectively induce tumor cell death only in cancer patients.

What is claimed is:

1. A method for inhibiting growth of a tumor in a patient, wherein the tumor comprises target cells, wherein the target cell comprises three or more target nucleic acids in its genome,
the method comprises:
administering a CRISPR/nCas composition for causing targeted cell death, and a PARP inhibitor to the patient,
wherein, the CRISPR/nCas composition for causing targeted cell death causes single-strand breaks at three or more target nucleic acids in the genome of the target cell,
the CRISPR/nCas composition for causing targeted cell death and the PARP inhibitor act together to induce cell death,
wherein, the CRISPR/nCas composition for causing targeted cell death comprises one or more types of CRISPR/nCas compositions,
wherein each type of CRISPR/nCas composition comprises:
a Cas protein having nickase activity, or a nucleic acid encoding the Cas protein; and
a guide RNA comprising a guide domain and a scaffold, or a nucleic acid encoding the guide RNA;
wherein, the scaffold interacts with the Cas protein such that the guide RNA forms a complex with the Cas protein, and
the guide domain of the guide RNA targets one of the nucleic acid sequences of the target nucleic acids.

2. The method of claim 1, wherein, the target cell comprises at least three target nucleic acids having different nucleic acid sequences from each other, and
wherein the CRISPR/nCas composition for causing targeted cell death comprises at least three types of CRISPR/nCas compositions,
the guide domain of the guide RNA of each type of CRISPR/nCas composition targets different nucleic acid sequence from each other,
the guide domain of the guide RNA of each type of CRISPR/nCas composition targets one of the nucleic acid sequences of the target nucleic acids.

3. The method of claim 1, wherein, the target cell comprises at least three target nucleic acids having the identical nucleic acid sequence,
the CRISPR/nCas composition for causing targeted cell death comprises at least one type of CRISPR/nCas compositions, and
the guide domain of the guide RNA of each type of CRISPR/nCas composition targets the nucleic acid sequence of the target nucleic acids.

4. The method of claim 1, wherein, each type of CRISPR/nCas composition is in a form selected from:
a composition comprising the Cas protein having nickase activity and the guide RNA, wherein the Cas protein binds to the guide RNA to form an RNA-protein complex; or
a composition comprising an mRNA encoding the Cas protein having nickase activity, and the guide RNA.

5. The method of claim 1, wherein, the PARP inhibitor is selected from i) Olaparib; ii) Rucaparib; iii) Niraparib; iv) Veliparib; v) Iniparib; or combination of any of i) to v).

6. The method of claim 1, wherein, the target nucleic acids are double-stranded DNA comprising a target strand and a non-target strand, and
the targeting one of the nucleic acid sequences of the target nucleic acids by the guide domain of the guide RNA of the CRISPR/nCas composition, is selected from:
a guide domain having an RNA sequence equivalent to one of DNA sequences of the non-target strand of the target nucleic acids;
a guide domain being capable of hybridizing with one of DNA sequences of the target strand of the target nucleic acids; or
a guide domain having an RNA sequence complementary to one of DNA sequences of the target strand of the target nucleic acids.

7. The method of claim 1, wherein, the CRISPR/nCas composition for causing targeted cell death and the PARP inhibitor are each independently administered to the patient.

8. The method of claim 1, wherein the target cell is a cancer cell, and the target nucleic acids are cancer cell-specific target nucleic acids.

9. A method for inhibiting growth of a tumor in a patient, wherein the tumor comprises target cells, wherein the target cell comprises a first target nucleic acid, a second target nucleic acid, and a third target nucleic acid in its genome,
wherein the method comprises:
delivering a CRISPR/nCas composition for causing targeted cell death, and a PARP inhibitor to the target cell,
wherein, the CRISPR/nCas composition for causing targeted cell death comprises:
a first CRISPR/nCas complex comprising a first nCas protein having a nickase activity, and a first guide RNA;
a second CRISPR/nCas complex comprising a second nCas protein having a nickase activity, and a second guide RNA; and
a third CRISPR/nCas complex comprising a third nCas protein having a nickase activity, and a third guide RNA;
wherein, a guide domain of the first guide RNA targets the first target nucleic acid,
a guide domain of the second guide RNA targets the second target nucleic acid, and
a guide domain of the third guide RNA targets the third target nucleic acid, and
wherein, the first CRISPR/nCas complex causes single-strand breaks at the first target nucleic acid,
the second CRISPR/nCas complex causes single-strand breaks at the second target nucleic acid,
the third CRISPR/nCas complex causes single-strand breaks at the third target nucleic acid, and
wherein, the CRISPR/nCas composition and the PARP inhibitor act together to induce the target cell death.

10. The method of claim 9, wherein, the first target nucleic acid, the second target nucleic acid, and the third target nucleic acid have different nucleic acid sequences from each other.

11. The method of claim 9, wherein, the first target nucleic acid has identical nucleic acid sequence with the second target nucleic acid; and
the first target nucleic acid has different nucleic acid sequence from the third target nucleic acid.

12. The method of claim 9, wherein, the first target nucleic acid, the second target nucleic acid, and the third target nucleic acid have the identical nucleic acid sequence.

13. The method of claim 9, wherein the target cell is a cancer cell, and the first target nucleic acid, the second target nucleic acid, and the third target nucleic acid are cancer cell-specific target nucleic acids.

14. A method for inhibiting growth of a tumor in a patient, wherein the tumor comprises target cells, wherein the target cell comprises a first target nucleic acid, a second target nucleic acid, and a third target nucleic acid in its genome,
wherein the method comprises:
delivering a CRISPR/nCas composition for causing targeted cell death, and a PARP inhibitor to the target cell,
wherein, the CRISPR/nCas composition for causing targeted cell death comprises:
a first CRISPR/nCas composition comprising an mRNA encoding a first nCas protein having nickase activity, and a first guide RNA;
a second CRISPR/nCas composition comprising an mRNA encoding a second nCas protein having nickase activity, and a second guide RNA; and
a third CRISPR/nCas composition comprising an mRNA encoding a third nCas protein having nickase activity, and a third guide RNA;
wherein, the first CRISPR/nCas composition induces formation of a first CRISPR/nCas complex, in which the first nCas protein and the first guide RNA are combined, within the target cell,
the second CRISPR/nCas composition induces a formation of a second CRISPR/nCas complex in which the second nCas protein and the second guide RNA are combined within the target cell, and
the third CRISPR/nCas composition induces a formation of a third CRISPR/nCas complex in which the third nCas protein and the third guide RNA are combined within the target cell,
wherein, a guide domain of the first guide RNA targets the first target nucleic acid,
a guide domain of the second guide RNA targets the second target nucleic acid, and
a guide domain of the third guide RNA targets the third target nucleic acid, and
wherein, the first CRISPR/nCas complex causes single-strand breaks at the first target nucleic acid,
the second CRISPR/nCas complex causes single-strand breaks at the second target nucleic acid,
the third CRISPR/nCas complex causes single-strand breaks at the third target nucleic acid, and
the CRISPR/nCas composition and the PARP inhibitor act together to induce the target cell death.

15. The method of claim 14, wherein, the first target nucleic acid, the second target nucleic acid, and the third target nucleic acid have different nucleic acid sequences from each other.

16. The method of claim 14, wherein, the first target nucleic acid has identical nucleic acid sequence with the second target nucleic acid; and
the first target nucleic acid has different nucleic acid sequence from the third target nucleic acid.

17. The method of claim 14, wherein, the first target nucleic acid, the second target nucleic acid, and the third target nucleic acid have the identical nucleic acid sequence.

18. The method of claim 14, wherein the target cell is a cancer cell, and the first target nucleic acid, the second target nucleic acid, and the third target nucleic acid are cancer cell-specific target nucleic acids.


SEQUENCE LISTING (53 sequences)

SEQ ID NO: 1 - protein, 1368 aa, Streptococcus pyogenes [sequence omitted]

SEQ ID NO: 2 - protein, 1368 aa, synthetic construct
  source 1..1368: note = D10A variant of SpCas9
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, synthetic construct
  source 1..1368: note = H840A variant of SpCas9
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - RNA, 86 nt, synthetic construct
  source 1..86: mol_type = other RNA; note = Scaffold sequence of guide RNA for SpCas9
gtttcagagctatgctggaaacagcatagcaagttgaaataaggctagtccgttatcaacttgaaaaagtggcaccgagtcggtgc

SEQ ID NO: 5 - RNA, 86 nt, synthetic construct
  source 1..86: mol_type = other RNA; note = Scaffold of guide RNA for SpCas9
gtttaagagctatgctggaaacagcatagcaagtttaaataaggctagtccgttatcaacttgaaaaagtggcaccgagtcggtgc

SEQ ID NO: 6 - RNA, 76 nt, synthetic construct
  source 1..76: mol_type = other RNA; note = Scaffold of guide RNA for SpCas9
gttttagagctagaaatagcaagttaaaataaggctagtccgttatcaacttgaaaaagtggcaccgagtcggtgc

SEQ ID NO: 7 - RNA, 76 nt, synthetic construct
  source 1..76: mol_type = other RNA; note = Scaffold of guide RNA for SpCas9
gcaccgactcggtgccactttttcaagttgataacggactagccttattttaacttgctatttctagctctaaaac

SEQ ID NO: 8 - RNA, 82 nt, synthetic construct
  source 1..82: mol_type = other RNA; note = Scaffold of guide RNA for SpCas9
gttttagagctagaaatagcaagttaaaataaggctagtccgttatcaacttgaaaaagtggcaccgagtcggtgctttttt

SEQ ID NO: 9 - RNA, 59 nt, synthetic construct
  source 1..59: mol_type = other RNA; note = tracrRNA of SpCas9
tagcaagttaaaaaaggctagtccgttatcaacttgaaaaagtggcaccgagtcggtgc

SEQ ID NO: 10 - RNA, 12 nt, synthetic construct
  source 1..12: mol_type = other RNA; note = Direct Repeat of guide RNA for SpCas9
gttttagagcta

SEQ ID NO: 11 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 12 - protein, 1307 aa, synthetic construct
  source 1..1307: note = R1226A variant of AsCas12a
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 13 - protein, 7 aa, synthetic construct
  source 1..7: note = Nuclear Localization Signal
PKKKRKV

SEQ ID NO: 14 - protein, 16 aa, synthetic construct
  source 1..16: note = Nuclear Localization Signal
KRPAATKKAGQAKKKK

SEQ ID NO: 15 - protein, 9 aa, synthetic construct
  source 1..9: note = Nuclear Localization Signal
PAAKRVKLD

SEQ ID NO: 16 - protein, 11 aa, synthetic construct
  source 1..11: note = Nuclear Localization Signal
RQRRNELKRSP

SEQ ID NO: 17 - protein, 38 aa, synthetic construct
  source 1..38: note = Nuclear Localization Signal
NQSSNFGPMKGGNFGGRSSGPYGGGGQYFAKPRNQGGY

SEQ ID NO: 18 - protein, 42 aa, synthetic construct
  source 1..42: note = Nuclear Localization Signal
RMRIZFKNKGKDTAELRRRRVEVSVELRKAKKDEQILKRRNV

SEQ ID NO: 19 - protein, 8 aa, synthetic construct
  source 1..8: note = Nuclear Localization Signal
VSRKRPRP

SEQ ID NO: 20 - protein, 8 aa, synthetic construct
  source 1..8: note = Nuclear Localization Signal
PPKKARED

SEQ ID NO: 21 - protein, 8 aa, synthetic construct
  source 1..8: note = Nuclear Localization Signal
PQPKKKPL

SEQ ID NO: 22 - protein, 12 aa, synthetic construct
  source 1..12: note = Nuclear Localization Signal
SALIKKKKKMAP

SEQ ID NO: 23 - protein, 5 aa, synthetic construct
  source 1..5: note = Nuclear Localization Signal
DRLRR

SEQ ID NO: 24 - protein, 7 aa, synthetic construct
  source 1..7: note = Nuclear Localization Signal
PKQKKRK

SEQ ID NO: 25 - protein, 10 aa, synthetic construct
  source 1..10: note = Nuclear Localization Signal
RKLKKKIKKL

SEQ ID NO: 26 - protein, 10 aa, synthetic construct
  source 1..10: note = Nuclear Localization Signal
REKKKFLKRR

SEQ ID NO: 27 - protein, 20 aa, synthetic construct
  source 1..20: note = Nuclear Localization Signal
KRKGDEVDGVDEVAKKKSKK

SEQ ID NO: 28 - protein, 17 aa, synthetic construct
  source 1..17: note = Nuclear Localization Signal
RKCLQAGMNLEARKTKK

SEQ ID NO: 29 - DNA, 4104 bp, synthetic construct
  source 1..4104: mol_type = other DNA; note = Optimized codon of SpCas9 D10A
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctactggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 30 - DNA, 4104 bp, synthetic construct
  source 1..4104: mol_type = other DNA; note = Optimized codon of SpCas9
atggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgac

SEQ ID NO: 31 - RNA, 20 nt, synthetic construct
  source 1..20: mol_type = other RNA; note = Guide domain of guide RNA for Umix-4
ctagaatgaccagtcaacag

SEQ ID NO: 32 - RNA, 20 nt, synthetic construct
  source 1..20: mol_type = other RNA; note = Guide domain of guide RNA for Umix-4
gctacctaagcacagccaca

SEQ ID NO: 33 - RNA, 20 nt, synthetic construct
  source 1..20: mol_type = other RNA; note = Guide domain of guide RNA for Umix-4
atggacccctaggtctccgg

SEQ ID NO: 34 - RNA, 20 nt, synthetic construct
  source 1..20: mol_type = other RNA; note = Guide domain of guide RNA for Umix-4
tgtttggtgggtactcaccc

SEQ ID NO: 35 - RNA, 20 nt, synthetic construct
  source 1..20: mol_type = other RNA; note = Guide domain of guide RNA for Hmix-4
atattccacaggtatatgca

SEQ ID NO: 36 - RNA, 20 nt, synthetic construct
  source 1..20: mol_type = other RNA; note = Guide domain of guide RNA for Hmix-4
gttcatggccagaaggacga

SEQ ID NO: 37 - RNA, 20 nt, synthetic construct
  source 1..20: mol_type = other RNA; note = Guide domain of guide RNA for Hmix-4
tggaatgtgccacacagcac

SEQ ID NO: 38 - RNA, 20 nt, synthetic construct
  source 1..20: mol_type = other RNA; note = Guide domain of guide RNA for Hmix-4
ctgccttgtcatagctgtac

SEQ ID NO: 39 - RNA, 20 nt, synthetic construct
  source 1..20: mol_type = other RNA; note = Guide domain of guide RNA for MT-50
gcagaggcaggtggatcatg

SEQ ID NO: 40 - DNA, 21 bp, synthetic construct
  source 1..21: mol_type = other DNA; note = siRNA for BRCA2
  misc_feature 1..19: note = RNA
  misc_feature 20..21: note = DNA
ctggaaaaggaatacagtttt

SEQ ID NO: 41 - RNA, 20 nt, synthetic construct
  source 1..20: mol_type = other RNA; note = Guide domain of guide RNA for HPRT1
ctagaatgaccagtcaacag

SEQ ID NO: 42 - RNA, 20 nt, synthetic construct
  source 1..20: mol_type = other RNA; note = Guide domain of guide RNA for BCR
gctacctaagcacagccaca

SEQ ID NO: 43 - RNA, 20 nt, synthetic construct
  source 1..20: mol_type = other RNA; note = Guide domain of guide RNA for DNAJC6
atggacccctaggtctccgg

SEQ ID NO: 44 - RNA, 20 nt, synthetic construct
  source 1..20: mol_type = other RNA; note = Guide domain of guide RNA for PLXDC2
tgtttggtgggtactcaccc

SEQ ID NO: 45 - DNA, 58 bp, synthetic construct
  source 1..58: mol_type = other DNA; note = Forward Primer of HPRT1
tcgtcggcagcgtcagatgtgtataagagacaggtgtacataaggatatacatataca

SEQ ID NO: 46 - DNA, 55 bp, synthetic construct
  source 1..55: mol_type = other DNA; note = Backward Primer of HPRT1
gtctcgtgggctcggagatgtgtataagagacagcatacctttccagttaaagtt

SEQ ID NO: 47 - DNA, 53 bp, synthetic construct
  source 1..53: mol_type = other DNA; note = Forward Primer of BCR
tcgtcggcagcgtcagatgtgtataagagacaggagccatgctgctgtctgta

SEQ ID NO: 48 - DNA, 54 bp, synthetic construct
  source 1..54: mol_type = other DNA; note = Backward Primer of BCR
gtctcgtgggctcggagatgtgtataagagacagaggcctggaggagtcttagc

SEQ ID NO: 49 - DNA, 53 bp, synthetic construct
  source 1..53: mol_type = other DNA; note = Forward Primer of DNAJC6
tcgtcggcagcgtcagatgtgtataagagacagctggaggattctggggtttt

SEQ ID NO: 50 - DNA, 53 bp, synthetic construct
  source 1..53: mol_type = other DNA; note = Backward Primer of DNAJC6
gtctcgtgggctcggagatgtgtataagagacagagcgcaataggaaggaagg

SEQ ID NO: 51 - DNA, 53 bp, synthetic construct
  source 1..53: mol_type = other DNA; note = Forward Primer of PLXDC2
tcgtcggcagcgtcagatgtgtataagagacagaaaagagggcagaaggatgg

SEQ ID NO: 52 - DNA, 54 bp, synthetic construct
  source 1..54: mol_type = other DNA; note = Backward Primer of PLXDC2
gtctcgtgggctcggagatgtgtataagagacagaattgcccagaaaacacagg

SEQ ID NO: 53 - RNA, 20 nt, synthetic construct
  source 1..20: mol_type = other RNA; note = Guide domain of guide RNA for non-target
gctccgatcgggttacaaca